(19) United States
(12) Patent Application Publication
(10) Pub. No.: US 2024/0065619 A1
(43) Pub. Date: Feb. 29, 2024

(54) SLEEP PATTERN BREATHING DETECTION

(71) Applicant: Neurostim Technologies LLC, Wellesley, MA (US)

(72) Inventors: Hoo-min D. TOONG, Cambridge, MA (US); Terry SPURLING, Menlo Park, CA (US); Anthony WEI, Palo Alto, CA (US); William C. ALTMANN, Austin, TX (US); Vivian CHENG, San Jose, CA (US); Eric CHENG, San Jose, CA (US); Fenghua LU, Winchester, MA (US); Tiejun ZHANG, Boston, MA (US)

(21) Appl. No.: 18/454,411

(22) Filed: Aug. 23, 2023

Related U.S. Application Data
(60) Provisional application No. 63/374,455, filed on Sep. 2, 2022.
(60) Provisional application No. 63/373,272, filed on Aug. 23, 2022.
(60) Provisional application No. 63/373,267, filed on Aug. 23, 2022.
(60) Provisional application No. 63/373,266, filed on Aug. 23, 2022.

(51) Int. Cl. A61B 5/00; A61B 5/08; A61N 1/04; A61N 1/36; G16H 50/30

(52) U.S. Cl. CPC A61B 5/4818; A61B 5/0816; A61B 5/4836; A61B 5/6833; A61N 1/0456; A61N 1/3601; G16H 50/30

(57) ABSTRACT

Example inventions detect and treat an occurrence of an apnea event during a sleep period of a user. Example inventions receive data corresponding to nasal air pressure of the user and/or audio of the user during the sleep period. Example inventions determine patterns in the data, based on the patterns, detect the apnea event and, in response to the detecting, treat the apnea event.

### CROSS REFERENCE TO RELATED APPLICATIONS

This application claims priority to U.S. Provisional Patent Application Ser. No. 63/373,265 filed on Aug. 23, 2022, U.S. Provisional Patent Application Ser. No. 63/373,257 filed on Aug. 23, 2022, U.S. Provisional Patent Application Ser. No. 63/373,272 filed on Aug. 23, 2022 and U.S. Provisional Patent Application Ser. No. 63/374,455 filed on Sep. 2, 2022. The disclosure of each of the applications is hereby incorporated by reference.

### FIELD

Example inventions are directed to systems and methods for improving sleep by detecting the patterns of obstructive sleep apnea episodes.

### BACKGROUND INFORMATION

Obstructive sleep apnea affects the quality of sleep. Obstructive Sleep Apnea (“OSA”) is the intermittent occlusion of the upper airway (“UAW”), resulting in the reduction of airflow through the throat. This may be due to neuromuscular factors or anatomical causes. The muscles that keep the airway open when active can allow it to close when relaxed. An obstructed airflow causes imbalances in oxygen exchange, measurable in the hemoglobin of the blood.

The equipment used for individuals suffering from OSA includes detection of each episode of OSA, in order to apply some treatment to the individual to reduce the effects of such an OSA episode. Detection of an episode occurs once the episode has begun, when the distress of interrupted breathing has begun.

In the context of sleep and sleep quality, current medical processes provide the subject with an index referred to as the “Apnea/Hypopnea Index”, or “AHI”, which is a count of the total number of events of any type of apnea or hypopnea per hour, as manually scored by a sleep professional and calculated during a sleep period. Apnea and hypopnea differ in their effect upon SpO2 and other body systems. By combining both into a single index, subjects with identical AHI numbers can have very different health outcomes. The AHI parameter provides little insight into the related biometrics that affect sleep disturbances, nor into related co-morbidities that affect the severity of a person's apnea or hypopnea.

Polysomnography (PSG) records the subject's sleep typically for only one night. Variations in sleep habits and sleep disturbances are therefore not entirely recorded when the study extends over one, two or only a few nights. Similarly, PSG does not typically record sleep on nights separated from one another, such as once-per-week or once-per-month, or long-term studies on subjects. This limitation also constrains such data gathering. PSG signals are used for more than AHI events such as sleep stages, REM sleep (EOG), arousals (EEG), SpO2 desaturation, etc.

PSG provides the clinician with a suite of measured parameters, usually recorded through the client's sleep period. The complexity and nuances in the data require a professional reading of the results to provide an adequate report in the sleep disturbances. Due to the lack of experienced professional resources, the reading of a PSG study is expensive, usually requires several days to deliver to the client, and provides little closed-loop discussion of the results.

PSG delivers data on sleep at the end of the recording session or later. This after-the-fact delivery does not provide the opportunity to affect the subject's sleep during the recording of sleep data or to compare sleep data in real time to other physiological metrics.

PSG studies, whether at a clinic or at home, involve wearing uncomfortable or obstructive equipment. This limits their continued use by those who want or need to assess their sleep quality,

### BRIEF DESCRIPTION OF THE DRAWINGS

FIG. 1 is an Obstructive Sleep Apnea Detection and Stimulation System in accordance to example inventions.

FIGS. 2A-FIG. 2D are diagrams of OSA-Related Nerves and Structures.

FIG. 3 is a User and an Obstructive Sleep Apnea Detection and Stimulation System in accordance to example inventions.

FIG. 4 is a Polysomnography Setup.

FIGS. 5-9 are Summary Graphs from Sleep Apnea Studies.

FIG. 10 is a flow chart and system illustrating functionality of relying on air pressure measurement to detect sleep events in accordance with one example.

FIG. 11 a flow chart and system illustrating functionality of relying on audio measurement to detect sleep events in accordance with one example.

FIG. 12 illustrates prior art example timeline graphs of nasal airflow and SpO2 measurements during a PSG sleep study.

FIG. 13 illustrates a prior art example sleep period timeline with plots of SpO2 for apnea and hypopnea events.

FIG. 14 illustrates an example smart device user interface in accordance to example inventions.

FIG. 15 shows relationships among various co-morbidities and apnea.

FIG. 16 shows a first example of a device used to capture breathing data.

FIG. 17 shows a second example of a device used to capture breathing data.

FIG. 18 shows a third example of a device used to capture breathing data.

FIG. 19 shows an example flowchart for states in the user interface.

FIG. 20 shows an example flowchart with user log-in.

FIG. 21 shows an example subject interface soliciting data on the user.

FIG. 22 shows an example subject interface soliciting data on the environment during the sleep session.

FIG. 23 shows an example subject interface short report on the sleep experience.

Further embodiments, details, advantages, and modifications will become apparent from the following detailed description of the embodiments, which is to be taken in conjunction with the accompanying drawings.

### DETAILED DESCRIPTION

Example inventions are directed to the detecting and severity assessment of sleep disturbance related to apnea and hypopnea events. Example inventions combine a suite of biometric and health aspects from a user/subject into a set of metrics that more fully inform both the subject and the medical staff to the severity of the subject's apnea and/or hypopnea. The biometric calculations using either analytics or machine learning, or both, are placed on a timeline representing the subject's sleep period, thereby showing the relationships between the biometrics and the detected apnea and/or hypopnea events. Related indices are shown in parallel with the timeline data. Among the measurements displayed are metrics related to the relative importance of apnea events versus hypopnea events.

Example inventions are directed to an integrated system that is placed on the skin of the user and activated and used with or without the help of a medical professional. The integrated system includes hardware and software to monitor biometrics related to breathing, and optionally, a computing resource able to analyze the breathing data and send improved parametric values to the device on the user's skin to improve the detection capabilities for that specific user, in a closed-loop system. Example inventions include the monitoring of biometrics and the prediction of the onset of an OSA episode.

Example inventions acquire a subject's sleep data using a device configured as a flexible patch with sensors and computational capabilities, such as disclosed in U.S. Pat. No. 11,712,557, the disclosure of which is hereby incorporated by reference.

Example inventions gather data during the subject's sleep period and analyze it in real time, thereby providing the processes to detect physiological events that pose a stress or danger to the subject, such that the subject's sleep or sleep position may be directly affected through the method of annunciations or signals from the system. Examples include apnea periods longer than 10 seconds that cause a drop in blood oxygen saturation. Examples include repetitive apnea events or hypopnea events or both at a rate that similarly causes a drop in blood oxygen saturation.

Example inventions monitor the subject continuously and detect each apnea event within the first few seconds of the start of the event. This predictive detection is accomplished using machine learning or analytics, or both, applied to the data from the subject in real time. By detecting an event within, for example, 5 seconds, instead of delaying the marking of an event until it has passed the 10 second threshold defined for an “apnea event”, example inventions may react to the event such as notifying the subject, notifying a second person such as a sleep partner, notifying a doctor in a real-time connection, or affecting the sleeping subject directly to cut short the apnea event and reduce such an event's side effects.

Example inventions monitor and detect each apnea event and associated parameters including, for example, severity, desaturation, interval since the previous apnea event, etc. Example inventions monitor and detect each apnea event and may pass forward the recognitions of events in real time for the purpose of treatment of apnea. The treatment may be performed with any of a variety of protocols, devices or systems, such as an external hypoglossal neve stimulator system (e.g., an external patch that includes electrodes), implanted hypoglossal nerve stimulator systems from “Inspire”, “Nyxoah” and “Imthera”, and positive airway pressure devices, such as CPAP, APAP, BiPAP. The detection using this system is agnostic and universal and is not tied to a particular method of treatment. As such, this system may be added to an existing treatment system to replace more cumbersome, invasive or expensive apnea detection methods.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

Neck TNA Device

A method and system is disclosed that physicians may offer to their patients to detect an OSA episode in preparation for the use of neurostimulation to counteract the apnea, without the complex system of a conventional sleep study in a clinic. The method and system create one or more signals which may be utilized by treatment equipment without the need for additional sensing subsystems.

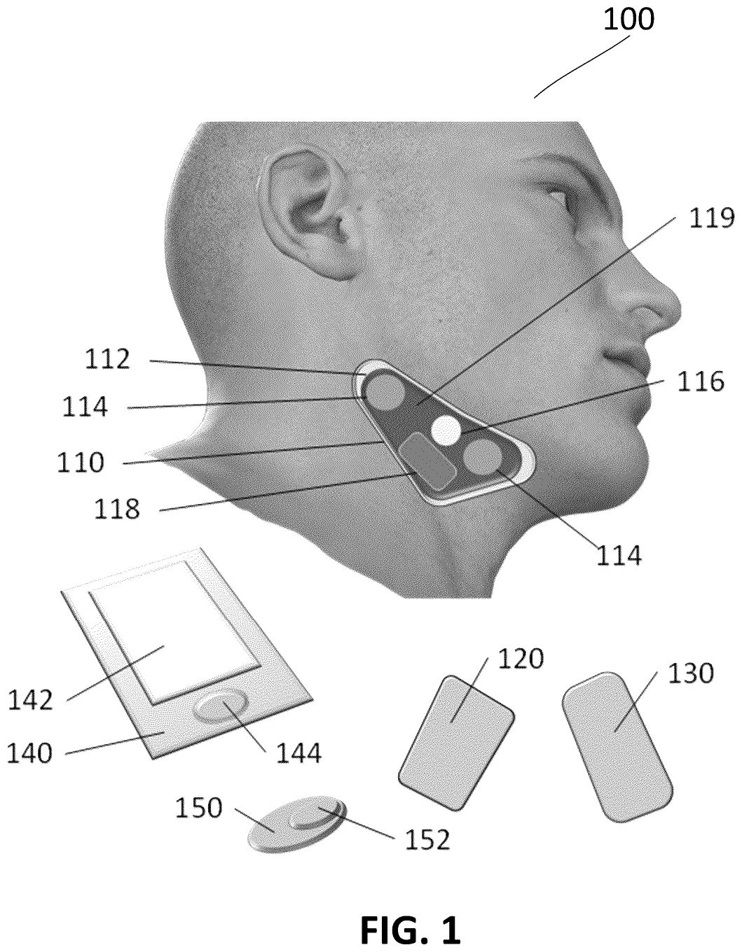

FIG. 1 is an illustration of components of an Obstructive Sleep Apnea Detection and Stimulation System 100, including a Neck Topical Nerve Activator (“TNA”) Device 110 (or “patch” 110), with a securing mechanism 112, and one or more electrode pairs 114 with each pair having a positive electrode and a negative electrode, and a power source 116, and a processor 118; and a Respiration Monitoring Device (“RMD”) 120; and an optional Posture Indication Device 130; and an optional Smart Controller 140, with a display 142 (e.g., a smartphone), and an acknowledgment button 144; and an optional Fob 150 with one or more buttons 152.

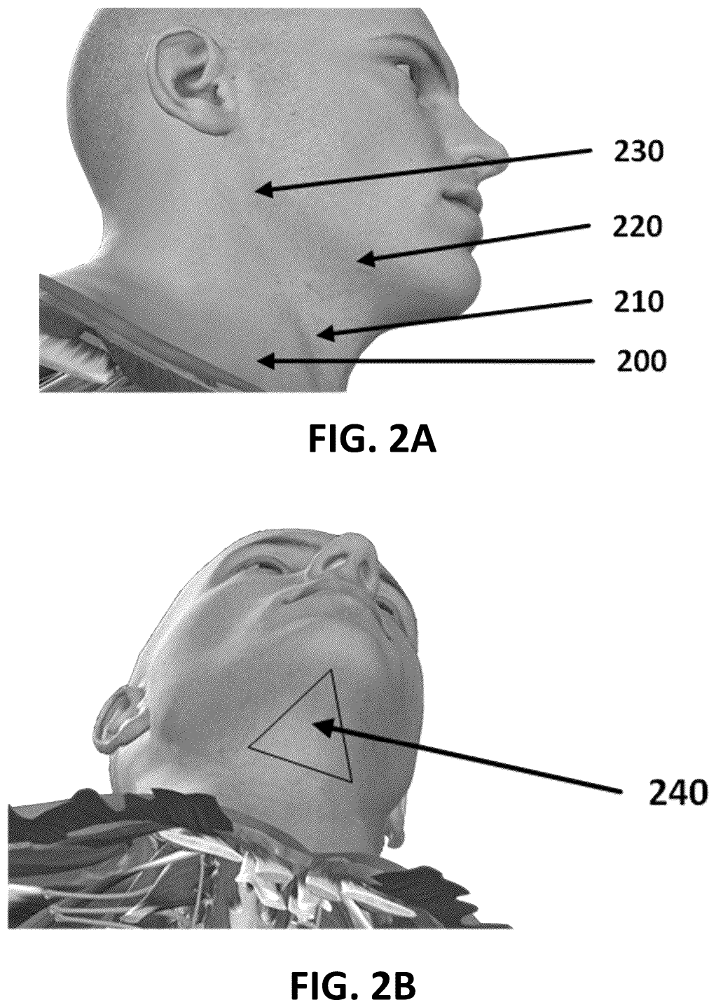

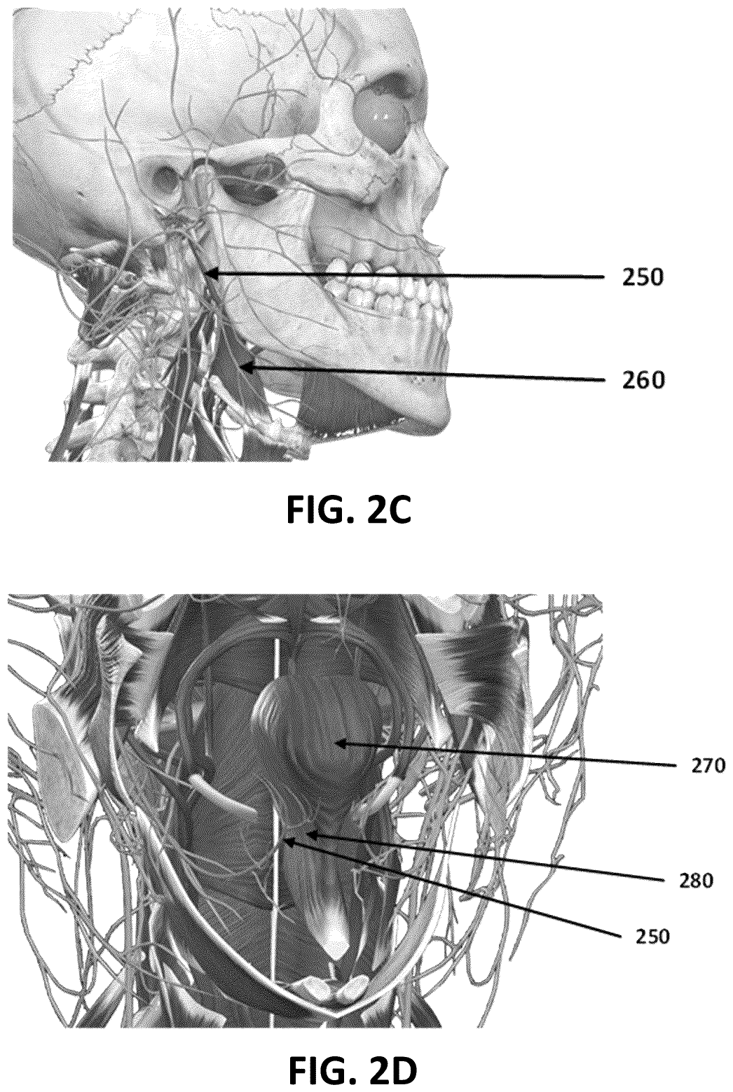

FIG. 2A shows a User 200, with a Neck 210, a Jaw Line 220, and an area behind the mandible 230.

FIG. 2B shows the User 200 and the submental triangle 240 below the chin.

FIG. 2C shows the User 200 and an internal view of the hypoglossal nerve 250 and the sublingual nerve 260.

FIG. 2D shows the User 200 and an internal view of the hypoglossal nerve 250, and the genioglossus muscle 280 under the tongue 270.

In FIG. 1, the Neck TNA Device 110 is in a shape to conform to a selected dermis surface to be electronically effective at stimulating the hypoglossal nerve 250 and to monitoring breathing. The Neck TNA Device 110 can be used for apnea detection as well as for delivering electrical stimulation. The Neck TNA Device 110 is electronically most effective for stimulation when the positive and negative electrodes are placed axially along the path of the nerve in contrast to transversely across the path of the nerve, which is not as electronically effective.

The Neck TNA Device 110 shape is designed to minimize discomfort for the User 200 when affixed in the target location.

The Neck TNA Device 110 includes one or more sensors that measure internal features or biometrics of the User in the neck area. These measurements are used to orient and place the Neck TNA Device most accurately in the target location and to monitor biometrics related to breathing. The sensor data is communicated to one or more of the Smart Controller 140, and the Fob 150; and is used by the Neck TNA Device.

In some examples, an indication such as LED or vibration is sent to the User to assist them in placing the Neck TNA Device.

TNA Device/patch 110 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 110 in one example provides topical nerve or tissue activation/stimulation on the user to provide benefits to the user, including treatment for OSA.

Patch 110 in one example can include a flexible substrate, a malleable dermis conforming bottom surface of the substrate including adhesive and adapted to contact the dermis, a flexible top outer surface of the substrate approximately parallel to the bottom surface, a plurality of electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip or discrete components and including an electrical signal generator integral to the malleable dermis conforming bottom surface configured to electrically activate the one or more electrodes, a signal activator coupled to the electrical signal generator, a nerve/tissue stimulation sensor that provides feedback in response to a stimulation of one or more nerves or tissues, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves or tissues of a user wearing patch 110 at least at one location proximate to patch 110. Additional details of examples of patch 110 beyond the novel details disclosed herein are disclosed in U.S. Pat. No. 10,016,600, entitled “Topical Neurological Stimulation”, the disclosure of which is hereby incorporated by reference.

FIGS. 2A and 2B show how the Neck TNA Device 110 is designed to be placed on either side of the neck, or on the jaw in one of several locations, such as the submental triangle 240, below the jawline 220, or behind the mandible 230; situated to accurately monitor biometrics related to breathing.

Detection Using Respiration Monitoring Device

The function of the Respiration Monitoring Device (“RMD”) 120 is to detect occurrences of interrupted breathing due to an apnea episode or hypopnea episode, and to notify one or more of the Neck TNA Device 110, or the Smart Controller 140, or the Fob 150 of such an episode.

OSA has traditionally been diagnosed through a sleep study, or polysomnography, usually performed in a clinic setting with multiple electrodes monitoring multiple body parameters. Requests for home screening tests are rising because of comfort and cost issues. The STOPBANG scale has been used to provide guidance to those individuals assumed to be at high risk for moderate to severe OSA, with the acronym defined as Snoring; Tiredness in daytime; Observation by third party of stopped breathing; high blood Pressure; Body Mass Index (BMI) greater than 35; Age over 50; Neck circumference greater than 16 inches; and male Gender. A STOPBANG score of 3 or more indicates a home test or sleep study should be performed, especially since up to 80% of people with OSA do not know they have apnea.

The RMD 120 includes one or more of accelerometers and an audio sensing device. The accelerometer detects rhythmic movements of respiration, or the lack of such movement. The audio sensing device detects the sounds of air flowing through the throat, snoring, body motion in bed, and background noise.

The RMD 120 captures data from one or more of its accelerometers and audio sensing device as a time-based series of measurements, and sends the data the Smart Controller 140, which modifies the data series to derive the power signature of movement data from the accelerometers and the power signature of audio data from the audio sensors. By filtering the data to remove power components outside the frequencies which are indicative of inspiration and expiration, the Smart Controller deduces the series of breaths in the data series.

An example, the Smart Controller 140 measures the frequency content across multiple distinct frequency bands in a continuous manner, recording that stream of measurement data in its memory. The data is analyzed in real-time to detect patterns of frequency content that match two or more formants, similar to formants characteristic of human speech. The formants are chosen from the full set of speech formants to use those which indicate tongue position. By matching the real-time signature in the breathing audio to the signatures of the formants, the Smart Controller marks times when the tongue moves from a forward position to a rear position in the oral cavity. When the Smart Controller detects that the tongue has moved or is moving into the airway, the Smart Controller signals to the Neck TNA Device 110 that an apnea episode has begun.

An example, the software converts the time domain audio signal into the frequency domain, such as by fast Fourier transform or digital Fourier transform or similar means, and creates a data sequence to show, at each sampled time step, the energy content of the audio at each specified frequency band.

An example, the frequency bands are determined at the beginning of the device usage from a default set.

An example, the frequency bands are determined at the beginning of the device usage during a training period, during which the device monitors the user and calculates the formant content for that specific user; followed by the diagnostic period, in which the audio data is distilled into formant energies according to the previously-determined formant definitions.

Following the previous example, the frequency bands continue to be adjusted during the diagnostic period, thereby using a closed-loop system to improve the distillation of apnea-related energies based on the real-time changes in the user's breathing, such as when the user changes sleeping positions, which may be detected by motion sensors on the device, or when the user's nasal or oral passageway changes as in congestion.

In some examples, the formant recognition is performed by firmware or software in the Neck TNA Device 110.

In some examples, the formant recognition is performed by firmware or software in the RMD 120.

In some examples the duration of each inspiration is measured by the RMD 120 to estimate the magnitude of the indrawn breath, the Obstructive Sleep Apnea Detection and Stimulation System 100 using this measurement to distinguish apnea events from hypopnea events. Thus, shorter, incomplete breaths will have a duration distinguishable from longer, complete breaths, using a threshold established by the Obstructive Sleep Apnea Detection and Stimulation System 100 or set and adjusted periodically by the Smart Controller 140, based on filtering of data to discern a minimum indrawn breath indication for the specific user.

The RMD 120 can be positioned at various locations on the body, depending upon which sensor and which body parameters are measured. For example, an RMD containing an accelerometer is positioned on the chest to detect rhythmic breathing patterns or on the neck in the submental region, or on the lower neck at the suprasternal notch; whereas an RMD containing an audio sensor can be positioned near the outside of the nasal passageway or on the neck in the submental region, or on the lower neck at the suprasternal notch.

In some examples, the RMD 120 is a separate unit from the Neck TNA Device 110, is positioned at various anatomical locations around the body away from the Neck TNA Device.

In some examples the RMD 120 is integrated within the Neck TNA Device 110, and monitors specific body signals at the same location as the Neck TNA Device.

Example Obstructive Sleep Apnea Detection and Stimulation System

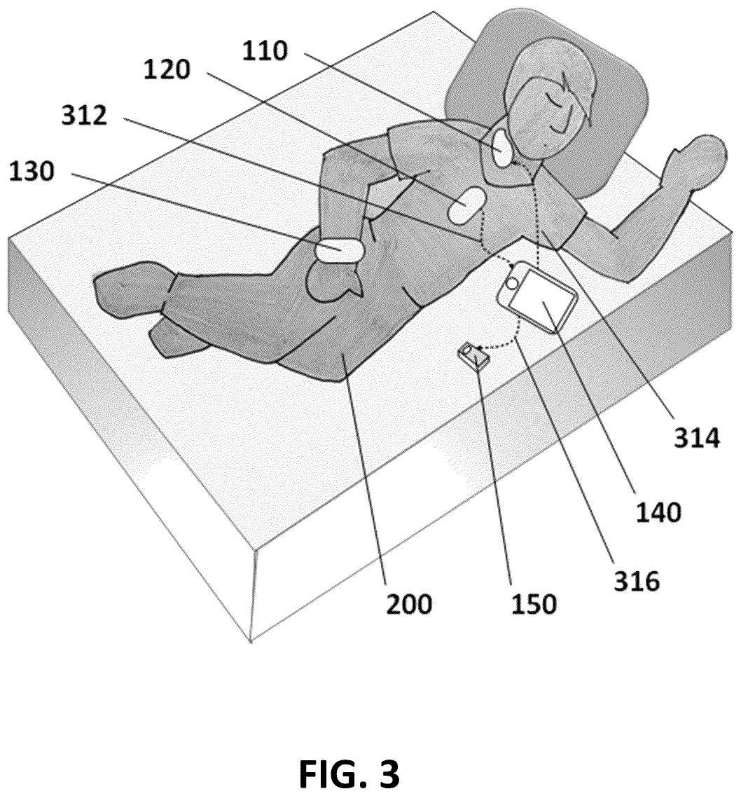

FIG. 3 illustrates an Obstructive Sleep Apnea Detection and Stimulation System 300 that includes a Neck TNA Device 110, and a Respiration Monitoring Device (“RMD”) 120. The RMD may be a separate unit or integrated within the Neck TNA. It may also include a Smart Controller 140 or Fob 150. The User 200 may indicate to the Neck TNA Device or Smart Controller or the Fob directly when the User is beginning a Sleep Period and again when the User is ending a Sleep Period. During the Sleep Period, when the RMD senses an OSA episode, the RMD then signals the Smart Controller or Fob that the apnea be recorded and optionally suppressed using the Neck TNA Device, then the Smart Controller signals to the Neck TNA Device to activate the nerve, then the Smart Controller optionally signals to the Fob to record such an activation event that the apnea is suppressed for a period of time.

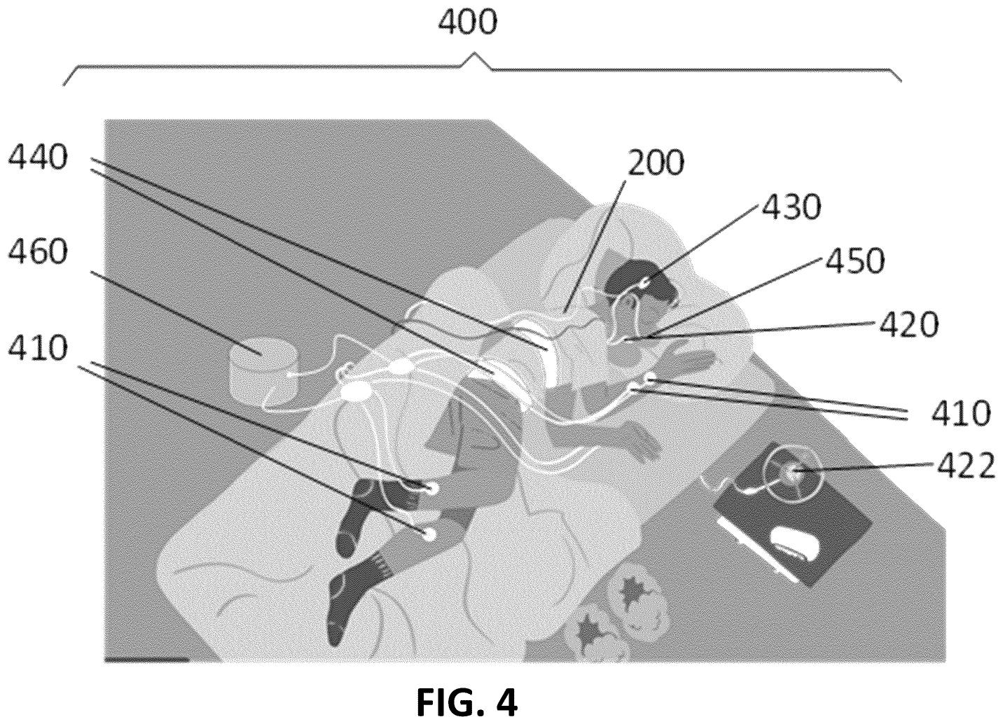

FIG. 4 illustrates a conventional polysomnography (“PSG”) system 400 that may include a number of sensors and wires attached to the User 200 while sleeping, including a set of Leg Movement Sensors 410 with wires, a Snoring Sensor Microphone 420, an Ambient Noise Microphone 422, an EEG Electrode 430, one or more Breathing Detection Belts 440, and an Airway Sensor 450. As may be seen in the figure, the wires connecting the sensors to the PSG Controller 460 may interfere with the User's sleep, or the measurements may be adversely affected by User movement, or the measurements may be stopped due to disconnection of one or more sensor due to User movement.

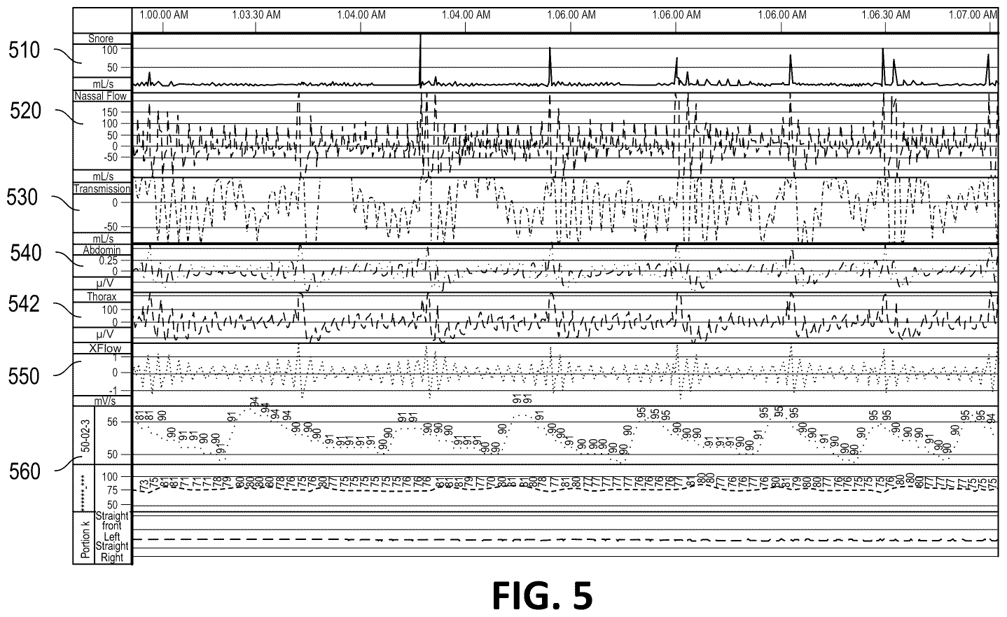

FIG. 5 illustrates an example of signals captured during a sleep period. Snoring is detected along Snoring Signal 510; Breathing is detected along Nasal Flow Signal 520, and Thermistor Signal 530; User's movement is detected along Abdomen Signal 530, and Thorax Signal 540; respiratory airflow volume is detected along XFlow Signal 550, and SpO2 Oxygen Saturation Signal 560.

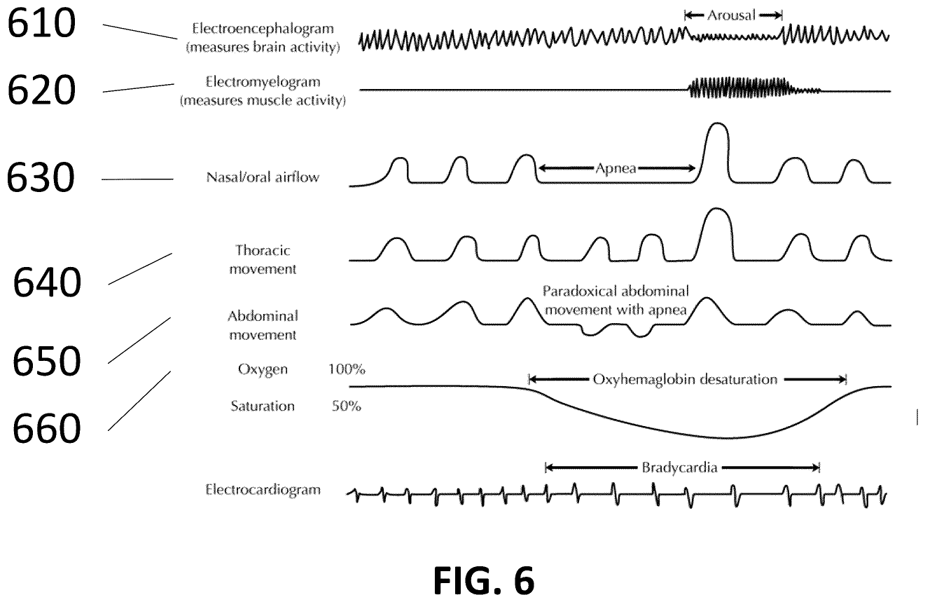

FIG. 6 summarizes measurements showing biometric data during a sleep study. The graph shows how only the Nasal/Oral Airflow Signal 630 indicates a sleep apnea event without trailing the sleep apnea event, such as is shown by the Oxygen Saturation Signal 660 and the EMG Signal 620 which are trailing signals. The other signals: the EEG 610, the Thoracic Movement Signal 640, and the Abdominal Movement Signal 650—do not distinctively indicate a sleep apnea event.

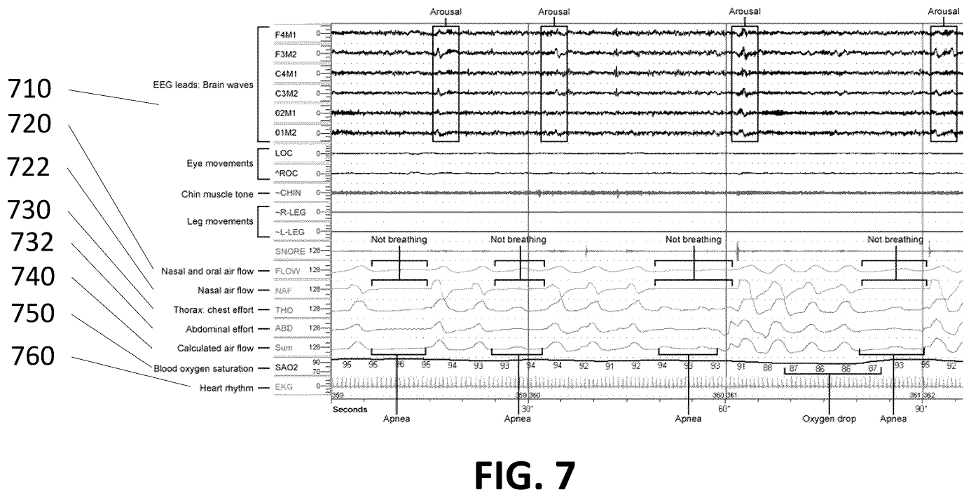

FIG. 7 summarizes measurements showing biometric data during a sleep study. The graph shows how airflow, measured by the Nasal and Oral Air Flow Signal 720, the Nasal Air Flow Signal 722, the Thorax Chest Effort 730, the Abdominal Effort 732, and the Calculated Air Flow 740 all indicate a sleep apnea event without trailing the sleep apnea event as is shown by the EEG Signal 710. The other signals: the Blood Oxygen Saturation Signal 750, and the Heart Rhythm Signal 760—do not distinctively indicate a sleep apnea event.

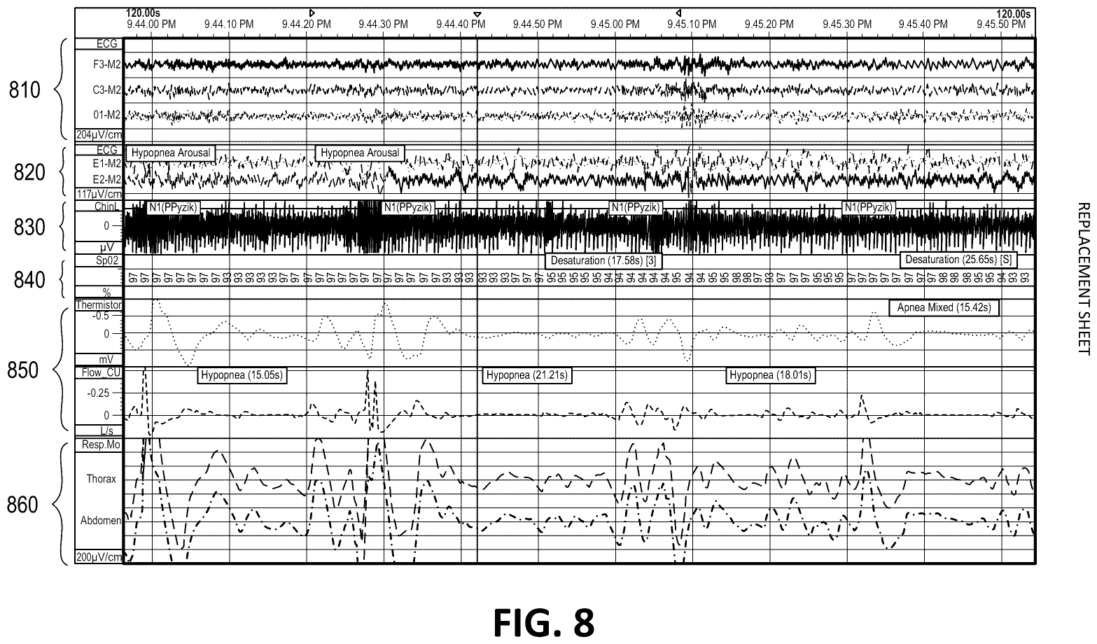

FIG. 8 summarizes measurements showing biometric data during a sleep study. Apnea events are strongly indicated coincident by Breathing Airflow Measurements 850, and somewhat indicated coincident by Breathing Effort Measurements 860; but either not clearly indicated or only indicated after the onset of a sleep apnea event by EEG Measurements 810, Electroculogram (“EOG”) Measurements 820, Electromyelogram Measurements 830, and Pulse Oximetry Measurements 840.

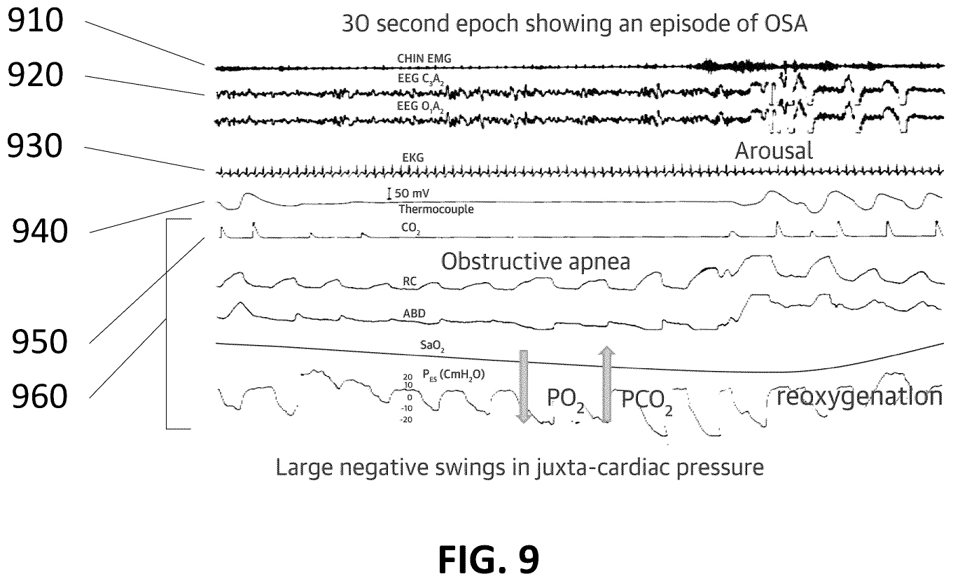

FIG. 9 summarizes measurements showing biometric data during a sleep study. Apnea events are only indicated after the event by EMG Measurements 910, EEG Measurements 920, EKG Measurements 930, and Blood Chemistry Measurements 960. The Thermocouple Measurements 940, from which air flow is calculated, and CO2 levels 950 correlate with coincident timing to sleep apnea events.

The graphs shown in FIG. 5 through FIG. 9 demonstrate that a measurement of breathing air flow is sufficient to indicate a sleep apnea event at a time coincident with the event and not trailing in time after the event has begun.

Machine Learning or Analytics Based OSA Detection

As disclosed above, in the context of sleep and sleep quality, current medical processes provide the subject with an AHI reading, which is a count of the total number of events of any type of apnea or hypopnea per hour, as manually scored by a sleep professional and calculated during a sleep period. Apnea and hypopnea differ in their effect upon SpO2 and other body systems. By combining both into a single index, subjects with identical AHI numbers can have very different health outcomes. The AHI parameter provides little insight into the related biometrics which affect sleep disturbances, nor into related comorbidities which affect the severity of a person's apnea or hypopnea.

Polysomnography provides the clinician with a suite of measured parameters, usually recorded through the client's sleep period. The complexity and nuances in the data require a professional reading of the results to provide an adequate report in the sleep disturbances. Due to the lack of experienced professional resources, the reading of a PSG study is expensive, usually requires several days to deliver to the client, and provides little closed-loop discussion of the results.

In contrast, example inventions combine a suite of biometric and health aspects from the subject into a set of metrics which more fully inform both the subject and the medical staff as to the severity of the subject's apnea and/or hypopnea. The biometric calculations using either analytics or machine learning, or both, are placed on a single common timeline representing the subject's sleep period, thereby showing the relationships between the biometrics and the detected apnea and/or hypopnea events. Related indices are shown in parallel with the timeline data. Among the measurements displayed are metrics related to the relative importance of apnea events versus hypopnea events.

Example inventions analyze the suite of biometric data from the user's sleep, using one or both of algorithmic processes and machine learning processes to deduce patterns in the sleep and rate the severity of the sleep disturbances. Combining the various data sources in the system, example inventions circumvent the delays inherent in professional, clinical evaluation, and provide the user, through its user interface, the means to look at the calculations and correlations in a closed-loop manner. The user benefits by having a timely and comprehensive sleep analysis which has digested more types of data than a sleep clinician can analyze manually. Trust is developed between the system and the user through the presentation of specific data and analysis, for example on the user's smart device. In additional to analyzing the data, example inventions use explainable artificial intelligence (“XAI”) to engage the user through the presentation of data, for example by providing a “walk through” of the results, thereby eliminating the problem of lack of trust in a black box machine learning system. One or both of the algorithmic and machine learning processes discerns rhythms and patterns in the nasal air pressure data and/or the audio data to a level of detail not visible to the clinician.

The system shows to the subject and the medical staff the severity of the subject's sleep disturbances as the night's sleep recording is reviewed on the timeline. Rather than a simple, scaler index of sleep disturbance (i.e., AHI), the system provides time-based data and informs the subject and doctor. This may lead to modifications in the subject's sleep behavior.

Sleep Severity Index and Sp02

Many factors can affect sleep. Many factors directly or indirectly affect apnea and/or hypopnea. As a result of these influences, a simple measure of “events per hour” provides an incomplete view of the individual's quality of sleep.

Examples of directly influential factors on apnea and/or hypopnea are age, body mass index (BMI), neck size, gender, sleep position, diet and recently ingested food or drink. Examples of comorbidities which influence apnea and/or hypopnea are history of stroke, coronary heart disease, hypertension, diabetes, depression, migraine, and dementia.

The definitions of “apnea” and “hypopnea” center around the limitation in airflow during an attempted breath. An apnea event is marked during polysomnography as a reduction in airflow of at least 90% for a duration of 10 seconds or greater. A hypopnea event is marked as a reduction in airflow volume of at least 30% for a duration of 10 seconds or greater, which allows for a hypopnea event to be marked up to 89% reduction in airflow, thus allowing for misinterpretation of one or more event by a somnography technician,

In addition to tracking respiration using airflow to fulfill the criteria for ‘apnea’ and ‘hypopnea’ events, example inventions track multiple other biometrics in real-time. Examples of additional biometrics include one or more of the following: partial oxygen (SpO2), body temperature, heart rate, heart rate variation, electroencephalogram (EEG), electrocardiogram (ECG), electrooculography (EOG), or actigraphy.

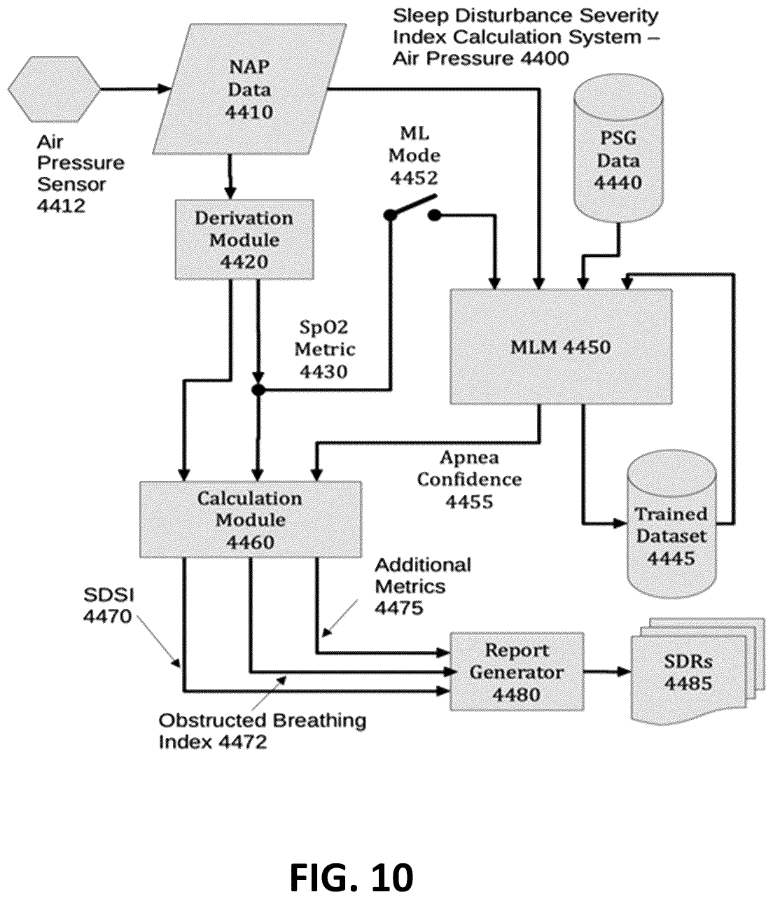

FIG. 10 is a flow chart and system illustrating functionality of relying on air pressure measurement to detect sleep events in accordance with one example. The sleep disturbance severity index calculation system—air-pressure (the air pressure based system) 4400 reports to the subject and the medical personnel a set of values based on the number of detected events, the periodicity of those events, the duration of the events and related parameters.

The air pressure based system 4400 of FIG. 10 includes nasal air pressure data (NAP Data) 4410 that is input from one or more air pressure sensors 4412, which may be implemented by patch 3010 with sensors, into both a derivation module 4420 and a machine learning module (MLM) 4450. Derivation module 4420 creates a data stream of a calculated SpO2 metric 4430. Polysomnography (PSG) data 4440 is input to MLM 4450 to train on labeled data and create a training dataset 4445. Correlation data 4455 from MLM 4450, operating on both training dataset 4445 and NAP data 4410 is input into a calculation module 4460. Other data from derivation module 4420 and SpO2 metric 4430 are also input to calculation module 4460.

Calculation module 4460 outputs one or more data streams as a sleep disturbance severity index (SDSI) 4470, an obstructed breathing index 4472, and additional metrics 4475. One or more of SDSI 4470 and additional metrics 4475 are input to a report generator 4480, which outputs sleep disturbance reports (SDRs) 4485. During the training phase, an ML mode 4452 disconnects calculation module 4460 from MLM 4450. During the detection phase, ML mode 4452 connects calculation module 4460 to MLM 4450 so that MLM 4450 may use zero or more of the calculated parameters as MLM inputs. Examples of MLM 4450 include Long Short-Term Model (LSTM) Recurrent Neural Networks (RNNs), and Convolutional Neural Networks (CNNs). The MLM 4450 is trained on the data in one or more polysomnography databases.

Air pressure based system 4400 collects data from a single sensor type and calculates the necessary parametric data to count breathing events, which include apnea and hypopnea events of all types. In one example, air pressure based system 4400 collects NAP data 4410, and converts the waveform through one or more stages of scaling, down-sampling and machine learning, to detect breathing events. Examples of sensors on the patch 3010 for the air pressure based system 4400 include air pressure sensor, air flow sensor and air temperature sensor. In embodiments that use a patch to sense the air parameters, the placement of the patch is near the air source (e.g., upper lip, nasal bridge, cheek, chin, etc.).

In one example, NAP data 4410 is converted into a derived signal which closely represents the partial oxygen in the sleeping user, SpO2 metric 4430. In one example, NAP data 4410 is converted into one or more metrics, such as rising edge rate of each breath, falling edge rate of each breath, breathing cadence, variability in breath cadence, volume of each breath which is calculated as the area under the curves of one or both of NAP data and SpO2 metric 4430.

Air pressure based system 4400 may direct its NAP data 4410 and derived SpO2 metric 4430 and some or all of the derived metrics into the machine learning module, MLM 4450. The conversion of NAP data into SpO2 data is based upon the proven correlation between the depth and duration of each breath and the duration between breaths which may represent a breath missed due to a sleep apnea event. The system and/or method determines the start and end of each breath from the processed sensor data and estimates the energy of each breath which is proportional to the breath's volume. As the subject fails to breath normally one or more times, the SpO2 decreases. With partial breaths, as for example in hypopnea, the subject's SpO2 also decreases, this drop directly related to the number of hypopnea breaths and time before full, regular breathing is restored. Thus, by determining the duration and spacing of each breath, the system and/or method determines a metric closely correlated to direct SpO2 measurement.

The MLM 4450 has been previously trained using as inputs the marked apnea and hypopnea events during the sleep periods of one or more persons, the NAF data for those recorded sleep periods and the SpO2 monitoring data for those recorded sleep periods. After the MLM is trained, it is used to establish correlations between the NAP and/or SpO2 data streams for the present subject and the derived metrics. This machine learning activity strengthens the significance of the detected breathing events by showing a correlation in time.

In one example, MLM 4450 establishes a correlation between NAP data 4410 and cadence of breathing, such that the obstructed breathing index 4472 represents more than simply the AHI. In one example, MLM 4450 establishes a correlation between NAP data 4410 and changes in volume of successive breaths, such that SDSI 4470 represents more than simply the AHI. In one example, MLM 4450 establishes a correlation between SpO2 data 4430 and duration of deoxygenation periods, such that SDSI 4470 represents more than simply the AHI.

An example, the estimation of SpO2 is calculated using deterministic models. An example, the estimation of SpO2 is determined by machine learning, following a training phase of the one or more machine learning models. An example, the estimation of SpO2 is determined by machine learning, following a training phase of the one or more machine learning models using data from a large database of sleep study individuals.

An example, the estimation of SpO2 is determined by machine learning, following a training phase of the one or more machine learning models using data from the individual subject, the subject having used one type of device which includes both audio sensor or sensors and a SpO2 sensor, the data collected and processed on this type of device thereby establishing a calibrated correlation between SpO2 and breath measurements, the subject using a different device for subsequent sleep periods, the device having only audio sensor or sensors. In the example, the subject uses the devices to accurately determine their sleep disturbances, having had to use a SpO2 sensor only when the circumstances of sleep change is noted.

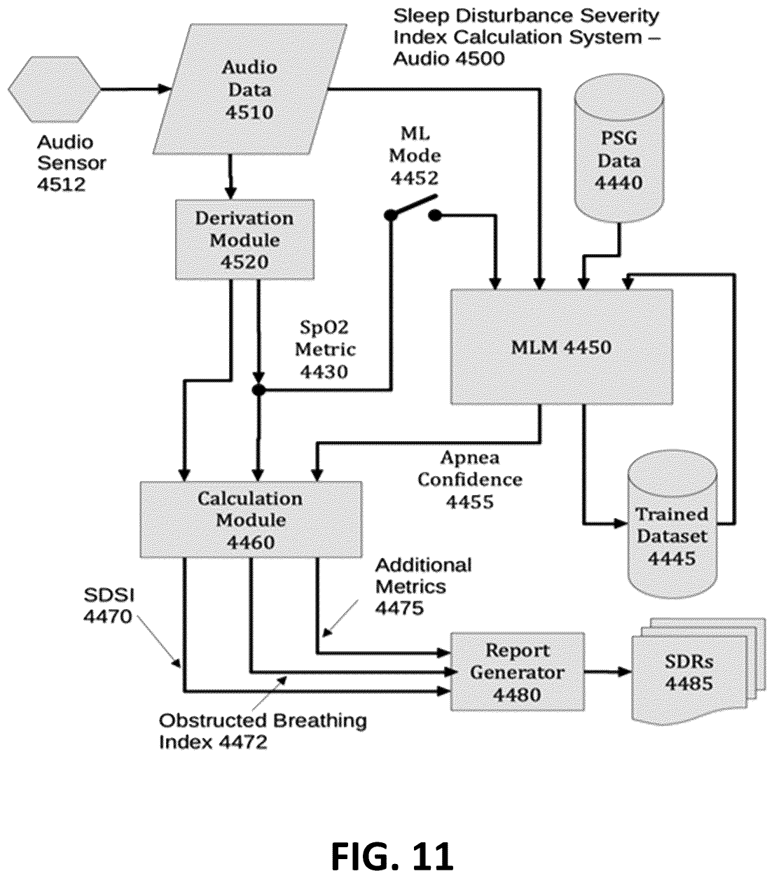

FIG. 11 a flow chart and system illustrating functionality of relying on audio measurement to detect sleep events in accordance with one example. In the Sleep Disturbance Severity Index Calculation System—Audio (the Audio-Based System) 4500, audio data 4510 is input from one or more audio sensors 4512 into both a derivation module 4420 and machine learning module (MLM) 4450. The data is processed in the same manner as in the Air Pressure Based System 4400, although with different algorithms and machine models. Examples of the audio-based system 4400 include a patch which includes audio sensors 4512 such as microphones, and placed close to the audio output sources.

During the training phase in each of the Airflow-Based System 4400, the Audio-Based System 4500, and similar sensor-based systems, the calculated biometrics and events are compared to the biometric and event data gathered during a PSG sleep study. For example, the list of apnea events and hypopnea events detected by the systems is compared to the sequence of similar events from the PSG sleep study in terms of onset, duration, intensity and other metrics. When the machine model or models have correlated the two sets of events and/or the two sets of biometrics, such as SpO2, then the one or more machine models are used to process sleep data from the user with confidence that the predicted events or biometrics will have matched similar observations or measurements during a PSG sleep study.

In both the Air Pressure Based System 4400 and the Audio-Based System 4500, there is only one parameter as input. From this single input stream, the systems derive the calculated event streams, output biometric data streams—such as SpO2—the SDSI 4470 and the obstructed breathing index 4472 metrics.

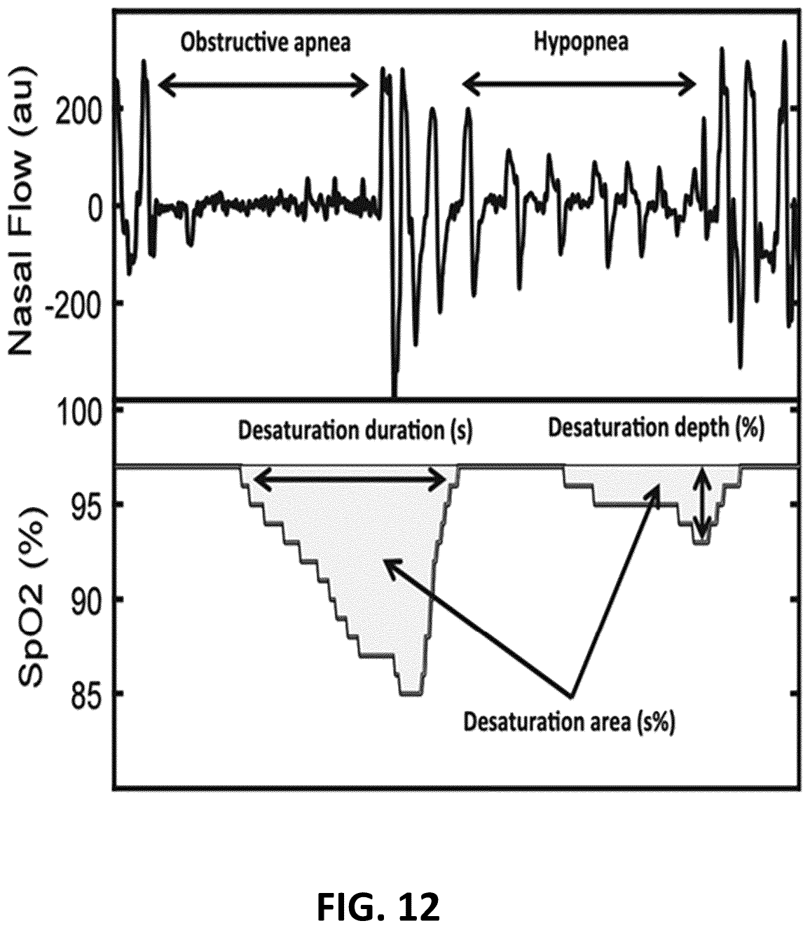

FIG. 12 illustrates prior art example timeline graphs of nasal airflow and SpO2 measurements during a PSG sleep study. The SpO2 level is reduced more during the course of an apnea event than during the course of a hypopnea event.

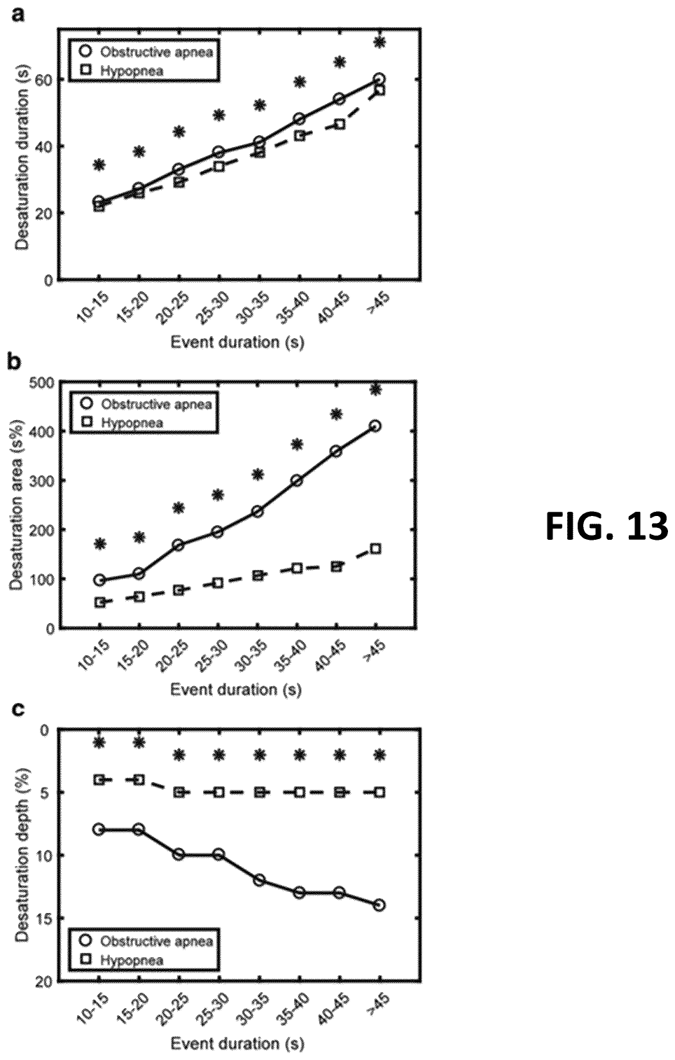

FIG. 13 illustrates a prior art example sleep period timeline with plots of SpO2 for apnea and hypopnea events. Median duration (a), area (b), and depth (c) of desaturations following obstructive apneas (circle) and hypopneas (square) in different event duration classes. Desaturations following obstructive apneas are longer, have higher areas, and are deeper compared to desaturations following hypopneas. Asterisks indicate statistically significantly different (p≤0.004) values between obstructive apneas and hypopneas, Mann-Whitney U test. As shown in FIGS. 12 and 13, apnea and hypopnea events are related to SpO2 differently. In a similar manner to modeling SpO2 based on audio signal, nasal airflow and/or nasal air pressure may be modeled based on audio signal such that a nasal airflow and/or nasal air pressure signals may be passed forward to other systems or subsystems so that those systems can be triggered using familiar signals.

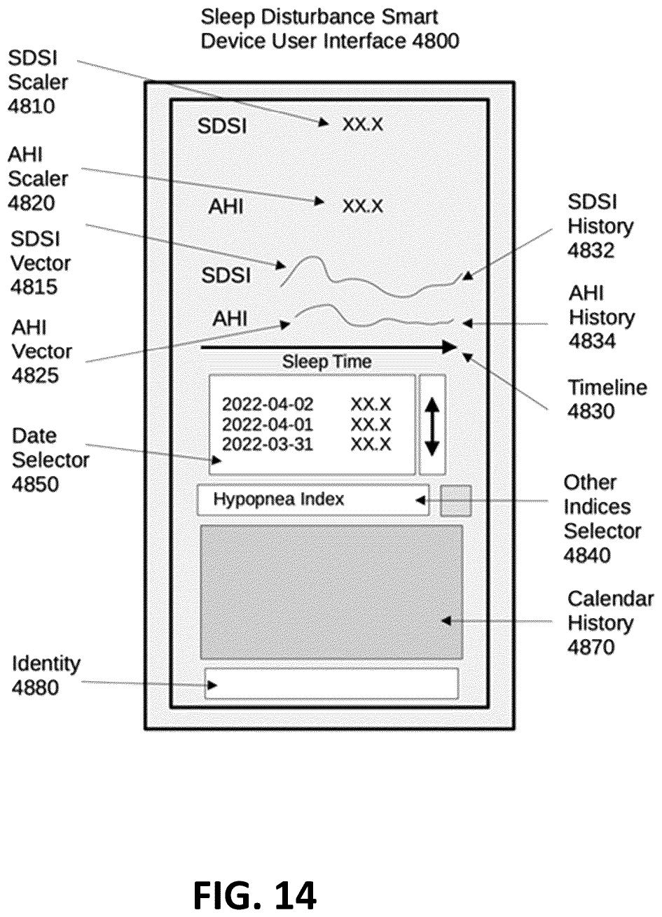

FIG. 14 illustrates an example smart device user interface 4800 in accordance to example inventions. UI 4800 displays the sleep severity indices in multiple ways to be most effective for the user. An SDSI scaler 4810 is a numerical value for the SDSI mean value across one night of sleep. An AHI scaler 4820 is a numerical value for the AHI mean value across one night of sleep. An SDSI vector 4815 is a curve plotted along the duration of a Timeline 4830 showing the SDSI as it changes. An AHI Vector 4825 is a curve plotted along the duration of a common timeline 4830 showing the SDSI as it changes. Each of 4815 and 4825 may be extended by selecting one or more dates from a date selector 4850. The SDSI history 4832 and AHI history 4834 may be expanded or contracted to show more or less detail along timeline 4830. User interface 4800 provides an other indices selector 4840, which may be a drop-down menu, to select other calculated or measured parameters. Exampled may be an hypopnea index, the SpO2 level, the sleep phase. Calendar history 4870 allows the user to select a range of dates from which a report may be generated. The user's personal Identity 4880 information is shown on the screen for convenience.

Because it is necessary to establish trust with the user that the results presented in UI 4800 are trustworthy, and because the apnea detection and treatment system is designed to modify the behavior of the user or clinician, the system must explain how it reached its conclusions in a clear and interpretable fashion. Parameters from the Derivation Module 4420 or from the Calculation Module 4460 can be used to explain the results of the MLM 4450. Further, the explainability of the MLM's conclusions will be used to elicit feedback from the user or clinician that will then modify the explanations of the current, or future, MLM results. This use of XAI with its iterative feedback loop that continually modifies the explanations of the MLM's results, while continually modifying the user's or clinician's behavior, is core to the manner in which the Apnea Detection and Treatment example inventions work.

In an example the specific apnea patterns being used to train the MLM can be presented to the user through the UI with percentages of concurrence with the user's own sleep signals to validate the veracity of the MLM's conclusions. This can be presented numerically or graphically through the UI.

In an example the number of occurrences of SpO2 values reaching below 90% during the sleep period, or normalized to a per hour basis, can be presented to the user as an explanation, in whole or part, for the MLM's conclusions. This can be done numerically or graphically through the UI.

In an example, data of parameters drawn from databases of similar subjects sleep recordings can be extracted, anonymized and presented to the user or clinician to demonstrate the conclusions from the MLM for this particular user.

Considering the deleterious effect of reduced blood oxygen, an apnea event has a larger effect on the body than a hypopnea event of the same duration. This is shown in FIG. 13, part C, where hypopnea events cause a reduction of 5% in SpO2 across the range of hypopnea event durations, while apnea events cause reductions of from 10% to 15% across the range of apnea event durations.

Air pressure based system 4400 and audio based system 4500 examine the input data and deduce parametric patterns in the data which provide insights into the sleep of the user. Some of these parametric patterns are so subtle as to avoid detection by the clinician. The patterns may be found during algorithmic analysis, looking forward and backward across time along the airflow and/or audio waveforms. The patterns may be found during machine learning analysis, after training, detecting correlations. Examples may include characterizing and categorizing the breaths immediately before and immediately after an apnea or hypopnea event, and accurate estimation of the SpO2 level based on the cumulative effects of multiple missing or weak breaths due to apnea or hypopnea event sequences. Examples include totaling and reporting the numbers of apnea events (central, obstructive and mixed) separately from the number of hypopnea events (central, obstructive and mixed), which informs the user more accurately than a single AHI value.

Systems 4400 and 4500 determine the one or more aspects of sleep disturbance severity index (SDSI) 4470 using a formula, weighting each of the influences of airflow data and/or audio data, calculated SpO2; and other parameters such as sleep position, comorbidities—for example:

$$SDSI = {\sum }_{t = start}^{t = end} ( {K}_{a} * {f}_{1} ( airflow ) ( + {K}_{b} * {f}_{2} ( audio ) + {K}_{c} * {f}_{3} ( Sp 02 ) ) + … + {K}_{y} * {f}_{4} ( position ) ( + {K}_{z} * {f}_{n} ( )$$

where, for example:

$${f}_{2} ( audio ) = {g}_{1} ( max ( amplitude ) ) + {g}_{2} ( \frac{energy}{breath} ) + {g}_{3} ( spectrumcontentprofile )$$

where ‘Kᵢ’ are weighting factors applied to each function in the calculation of SDSI; ‘fᵢ’ are the functions, each incorporating the influence of one or more data series, such as airflow or audio; ‘gᵢ’ are functions within the calculation of one of ‘fᵢ’, such as calculated data series for calculating the influence of audio data; ‘t’ is the time duration of the sleep period, such as measured in minutes or hours. SDSI 4470 may be presented as a set of one or more values representing the sleep disturbance index across the entire sleep period. SDSI 4470 may be presented as a series of sets of one or more values representing the sleep disturbance index across a portion of the sleep period, each set in the series corresponding to a period of minutes during the overall sleep period. For example, SDSI 4470 may be a series with each set of values in the series corresponding to a period of 10 minutes, such that a sleep period of 8 hours would have 48 series sets in the reported SDSI.

In examples, systems 4400 and 4500 use MLM 4450 as part of an end to end pipeline for apnea detection using a 1D Neural Network given input polysomnography data. The pipeline includes data preprocessing, automated “semi-supervised” data labeling, model training, and deployment.

For the data preprocessing, given input polysomnography (PSG) recordings, or input data that can also be sourced from sensors originating from other sources such as human subjects, the data is preprocessed in the following manner:
(1) Downsampling to 5 HZ to 400 HZ Hz to reduce model training time
(2) Two-step normalization:
   Compress entirety of signal to range [0,1]
   Normalization to make mean 0, standard deviation 1

For data labeling and segmentation, traditional machine learning setups require ground-truth labels associated with each data point (supervised classification), which may be challenging in situations where ground-truth annotations are not available. Instead, example inventions use a semi-supervised approach, learning from a small set of existing labels to manually generate pseudo-labels which are then used for training/validation/testing.

Example inventions use a variety of metrics (precision, recall, Pearson coefficient, Kappa coefficient) to measure how well the predicted events match up with the expert annotations. These generated labels (positive=apnea/hypopnea, negative=no apnea) are then used to curate the dataset for the machine learning model. In the case of class imbalance between the positive/negative classes, example inventions downsample the “majority” class so the number of data points in each class is approximately equal to prevent bias.

Example inventions use a one-dimensional (among several considered such as a long short-term memory (“LSTM”) and a convolutional neural network (“CNN”)) neural network, which was observed to yield better accuracy and faster training/inference speeds. While convolutional neural networks are traditionally suited towards image processing applications in computer vision; in recent years 1D neural networks have proven reliable for handling temporal univariate data.

Example inventions use a binary classification model which either predicts apnea (1) or no apnea (0). The model is trained on the dataset following a train/validation/test split in the ranges of 40-70%/10-30%/10-30% until the loss converges.

Example inventions measure the test accuracy on unseen data (data that the model has not been trained on), illustrating the model's generalization abilities to incoming data streams. Test accuracy is defined as the average % correct predictions across all test batches. Further, example inventions visualize a confusion matrix, which indicates the number of True Positive, True Negatives, False Positives, and False Negatives across all test data points. As new test points come in from human subject recordings, they are added to the training dataset and retrained on existing model's weights, creating a feedback loop to iterate and optimize the model further.

In examples, calculated metrics systems 4400 and 4500 or similar single-sensor-based systems may include periodicity of breathing events, duration of breathing events, breath volume for each breathing event, clustering of breathing events, times between clusters of breathing events, rate of inhalation, and the like.

In one example, calculated metrics in air pressure based system 4400 may include the slope of the first breath or breaths after a breathing event (quantifying ‘gasping’ breaths), differentiation of nasal breaths from oral breaths according to each breath's profile, and the like.

In one example, calculated metrics in audio-based system 4500 may include frequency domain energy content across a range of frequencies, distribution of frequency content across the span of each breathing event (such as higher pitch at the beginning of a ‘gasping’ breath), and the like.

In one example, systems 4400 and 4500 may include as inputs other factors or real-time observations such as sleep position, sleep phase, arousal events, background noise magnitude, and the like. One or more of these supplemental factors may be plotted on sleep disturbance index user interface 4800. SDSI 4470 and the obstructed breathing index 4472 metrics may be calculated without dependence on these supplemental factors. Correlation between one or more supplemental factor and the index or indices may be shown on the user interface, such as between SDSI 4470 and sleep position.

Comorbidity

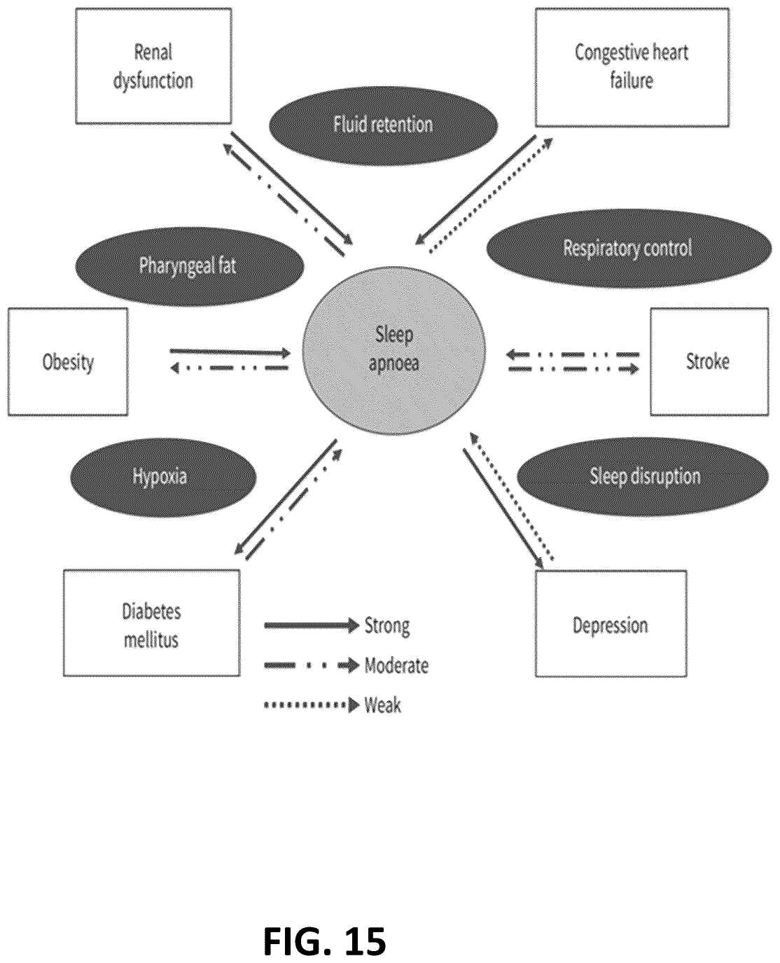

In example inventions, a report is sent to the subject, a second person, or a medical professional after the sleep period associations found through analysis between sleep disturbances and co-morbidities. FIG. 15 illustrates some of the morbidity aspects that are related to sleep apnea.

Machine learning or analytics or both are used along with biometric data on the current subject to confirm one or more comorbidity relationships that already exist in the subject. Examples are obesity, restless leg syndrome (RLS), periodic leg movement disorder (PLMD) and bruxism that is correlated to arousals associated with apnea.

Machine learning along with biometric data on the current subject is used to predict the probabilistic onset of one or more comorbidity relationships. An example is sleep apnea with heart disease or atrial fibrillation.

Using machine learning along with biometric data on the current subject can establish causality or reciprocity between sleep apnea and one or more comorbidity ailments. Examples are shown in FIG. 15 including diabetes mellitus, obesity, pharyngeal fat, fluid retention, hypoxia, renal dysfunction, stroke, congestive heart failure, depression, issues of respiratory control, that may be caused by or contribute to sleep apnea, and that may cause or contribute to sleep apnea.

An example, machine learning (ML) with one or more models is trained on PSG data from sleep study datasets. The ML model or models have hyperparameters that are adjusted using the training data. The ML model or models' hyperparameters may be further tuned for each individual subject using data from that subject's sleep periods. Sleep events are recognized using this real-time sleep apnea diagnostics.

An example, the presence of additional sensor or sensors on the system allows for concurrent measurement of other sleep parametrics, these then used in a closed-loop manner to improve the accuracy and robustness of the machine learning.

An example, the system and method combine machine learning or analytics or both based on a database from a population of persons with apnea and/or with one or more comorbidity ailments, in particular medical issues shared with the current subject, along with machine learning or analytics or both on the current subject's sleep data and biometric data on the current subject to confirm, predict or establish causality or reciprocity between sleep apnea and one or more comorbidity ailments.

An example, longitudinal studies are performed with a disposable patch because of its convenience and ease of at-home use. This is not done with Sleep Clinic Studies which rarely have more than one follow-up measurement several months/years apart; subjects can choose to monitor their apnea daily, weekly, monthly, or yearly with the device. These subject profiles can benefit from population studies of peers or other phenotypes over time, an extension not offered by conventional sleep labs nor by home sleep tests (HST).

An example, the system uses NAP/NAF analyses combined with electronic medical records (EMR) and/or electronic health records (EHR) (which are static) to gain insights as to cross disease conditions, comorbidities, and predictive upcoming conditions.

An example, ML approaches for disease diagnosis are calculated by performing breath pattern analysis over large populations and then correlating specific patterns with disease states through large populations of EHR/EMR Sleep apnea is just one example of breath pattern analysis that this ML/analytic approach can recognize. Other respiratory diseases can be diagnosed;

An example, the system's apnea analyses are used in critical care settings (e.g., ICU) to correlate with life-threatening conditions such as sepsis. It is important that the onset of sepsis be detected in real-time.

An example, the calculated breathing signal, together with ML and/or analytics, can detect/diagnosis asthma, COPD, and other respiratory conditions applying the same technique to training on databases of subjects with these conditions.

Detecting Device

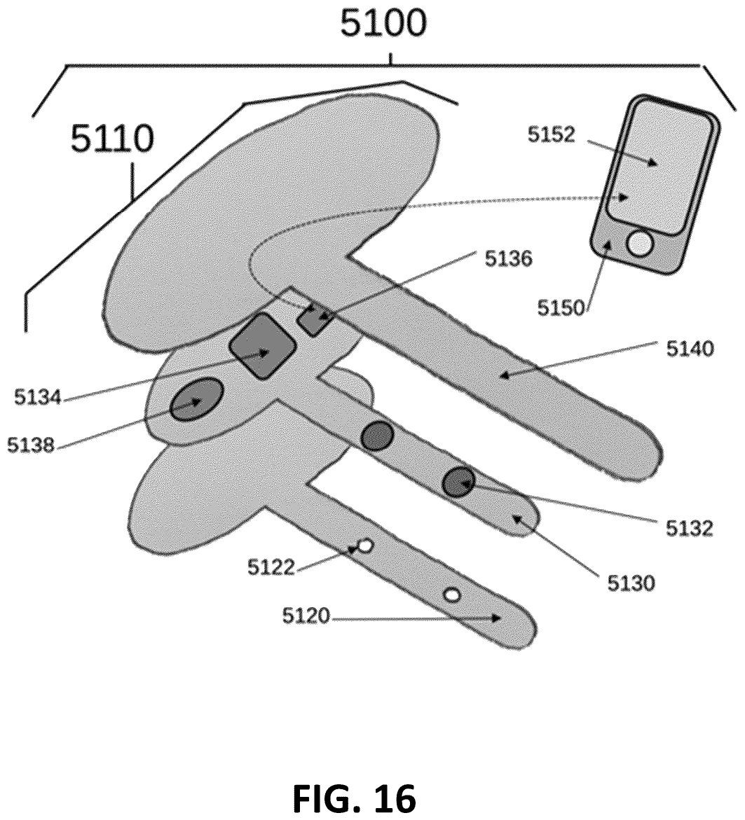

As disclosed, example inventions implement a patch which can include sensors as a device for detecting OSA. FIG. 16 illustrates an example System 5100, which includes a patch/Device 5110 and a Smart Device 5150. The Device is affixed to the skin with Adhesive 5120 such that the Device may be removed after a period of sleep without damaging the skin. This is especially important for skin on the face and nose, and more so for older subjects. The Device includes a Substrate 5130, one or more Sensor 5132, a Processor 5134, a Wireless Connection 5136, a Power Source 5138 (e.g., a battery), and a Padding 5140. The Device connects across the Wireless Connection to the Smart Device, which provides a User Interface (UI) 5152. The Substrate is constructed of a material thin enough and flexible enough to support the electronics and Power Source as well as to conform to the subject's skin shape. The Processor receives data from the Sensors and sends data out through the transmitter portion of the Wireless Connection. The Wireless Connection receives data from the Processor and from the Smart Device to which the Device is connected by wireless means. The Wireless Connection transmits data received from the Smart Device to the Processor and data received from the Processor to the Smart Device. The Padding protects the circuitry and the Power Source from interference by the subject or damage in use. The edges of the Padding are smooth to prevent injury when handled or worn by the subject. The Sensor or Sensors are included in the circuitry to collect data from the subject such as audio while breathing.

FIG. 16 shows an example layered stack-up for the Device 5110. The Adhesive layer 5120 is affixed to the bottom of the Substrate layer 5130 and the Padding layer 5140 is affixed to the top of the Substrate. The electronics are confined within, isolated from the subject's skin as well as from moisture and contamination.

The Device 5110 includes multiple components as shown by example in FIG. 16. The usefulness of the Device may be extended by allowing the subject to replace various components of the Device. An example, the subject may remove and then replace the Power Source 5138 from a device. An example, the subject may remove and then replace the Adhesive 5120 from a device. An example, the subject may separate the recyclable components from the non-recyclable components of a device following instructions provided to the subject, discarding each in the appropriate manner.

The Device 5110 may be provided in a variety of shapes to accommodate the variations in the dimensions and sensitivity of the subject's skin.

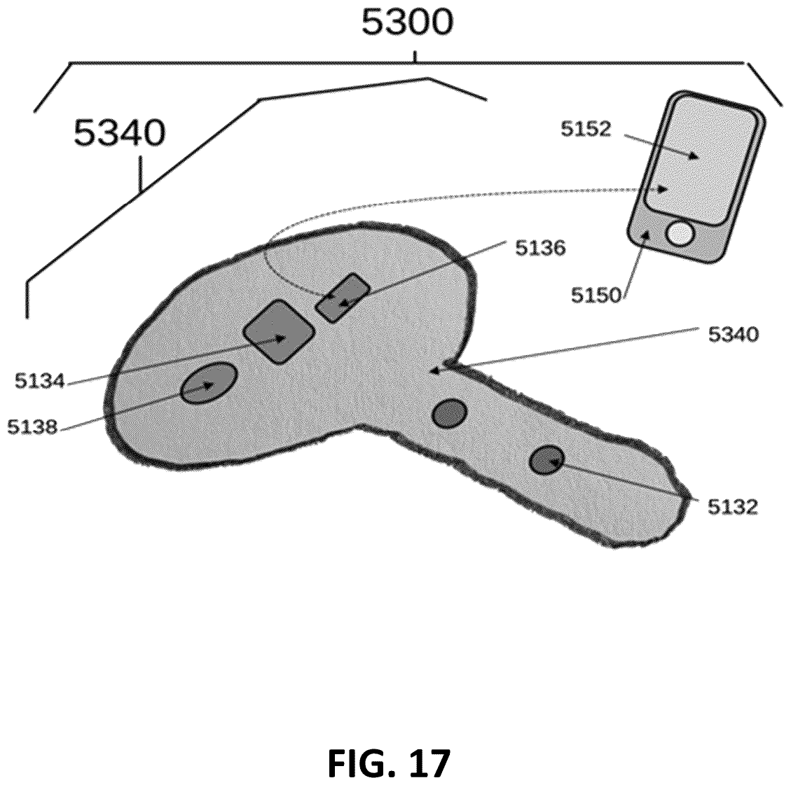

FIG. 17 shows an example device 5300 that has a single-sided shape 5310 to be placed on one side of the bridge of the nose and on the cheek. The wider end contains one or more component, such as Battery, sensors, Processor. The narrower portion of the is placed across the bridge of the nose.

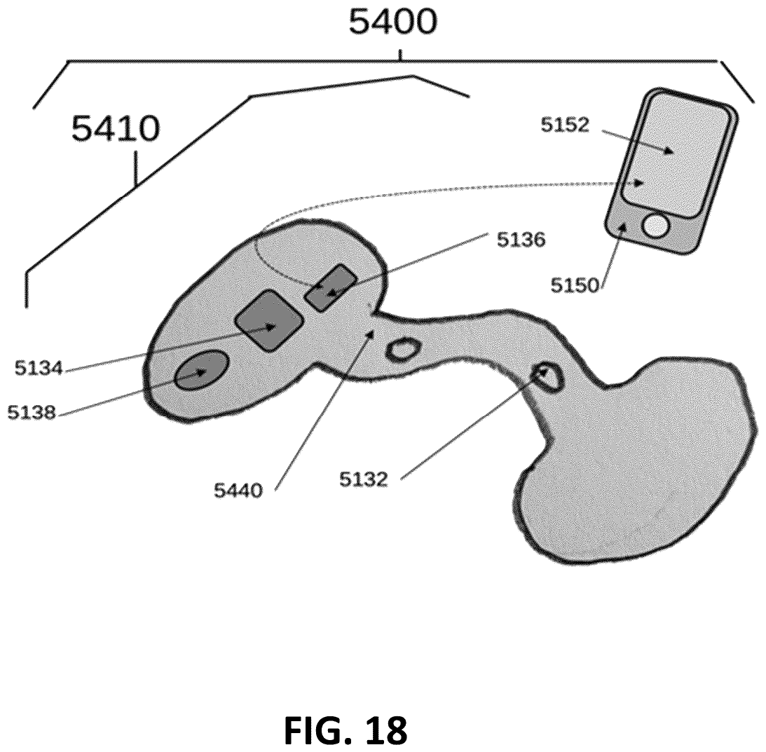

FIG. 18 shows an example device 5400 that has a dumbbell shape 5410, with a narrow section between the two wider ends, the narrow portion to be placed across the bridge of the noise, and each wider section to be placed on one cheek.

An example, device 5410 may be of a dumbbell shape but with each of the left and right side having different shapes. An example, device 5410 may be of a dumbbell shape but with each of the left right side having triangular or polygonal shapes, designed to fit properly in the space provided on the left and right cheek while connected across the bridge of the nose.

An example, a device contains additional sensors.

An example, a device has dimensions which fit it onto the bridge of the nose without extending onto either the left or right cheek, the dimensions being adequate to contain all components of the Device.

The subject may have trouble placing the Device 5110 on the face accurately. The Device includes markings or extensions of the Substrate and/or padding to assist the subject in proper placement. An example, a device has on its top surface one or more markings, such as arrows or lines or icons, which together with the instructions provided to the subject assist the subject in proper placement of a device.

An example, a device has on the perimeter of its substrate and/or padding extended shapes, such as tabs or prongs, which together with the instructions provided to the subject assist the subject in proper placement of a device.

An example, the smart device includes a camera and a display, and the software provided to the subject allows the subject to photograph the placement of the Device after placement on the face, this software working with the camera or cameras to verify proper placement relative to facial features of the subject and provide feedback to the subject in case adjustments are necessary for proper placement.

The dimensions of subject's faces vary. The Device 5110 is offered in multiple sizes to accommodate this variation in size and shape. An example, a device is provided in multiple sizes which vary the length and/or width of the portion designed to be placed across the bridge of the nose.

An example, a device is provided in multiple sizes which vary the dimensions of the left- and/or right-side of the device, thereby accommodating different facial structures, providing the means to avoid placement on wounds or irregularities on the skin on the left and/or right side.

An example, a device has a Substrate and Padding which may be peeled away by the subject around a portion of the perimeter thereby changing the overall dimensions of all or part of the device to accommodate variations in facial dimension.

An example, a device has an asymmetrical shape such that it may be worn in two orientations, reversing the left and right side so that the two sides of different dimensions are placed most comfortably on the face.

An example, a device is designed to be placed wholly or partially inside one or both nasal passages, one of more of its sensors positioned to acquire data from locations at the nasal opening or inside the nasal passage. The shape of the substrate, the padding and the adhesive is designed to fit comfortably in or near one or both nasal openings, and to secure it in place, and to provide means for comfortable removal. For example, one or more of the sensors measures one or more of nasal air pressure, nasal air pressure, air temperature and/or humidity.

An example, a device is designed to be placed near the mouth opening, such as on the lips, below or above the lip, on the chin, on the throat, one of more of its sensors positioned to acquire data. The shape of the substrate, the padding and the adhesive is designed to fit comfortably near the mouth, on the throat or chin, and to secure it in place, and to provide means for comfortable removal. For example, one or more of the sensors measures one or more of nasal air pressure, nasal air pressure, air temperature and/or humidity.

An example, a device is comprised of two or more subsystems, each subsystem having a substrate, padding, adhesive and power supply, to be placed on the skin, one or more subsystem having a processor, one or more subsystem having one or more sensor, the sensor or sensors communicating wirelessly to the one or more processor, all subsystems coordinating their communication and data flows and one or more subsystem sending and receiving data to and from the smart device.

An example, a smart device has a sensor of type complementary to the sensor or sensors on the device, the combination of sensors on the smart device and on the device collecting data to be analyzed to provide improved results over the use of one or more sensors on the device alone.

An example, a device includes circuitry for processor, sensor or sensors, power supply, wireless connection is incorporated into a facially mounted device such as a cosmetic applique or mask, facial mask, sheet mask, hydrating mask.

An example, a device includes circuitry for a subset of processor, sensor or sensors, power supply, wireless connection is incorporated into a facially mounted device such as a cosmetic applique or mask, facial mask, sheet mask, hydrating mark, the subset of circuitry connected to and working with the electronics on the circuitry on the mask.

An example, a device communicates using a wired connection to the smart device, such as a cable to a smart phone.

An example, the system is disposable.

An example, the system is reusable over more than one sleep period and then is disposable.

An example, a device collects a continuous series of data readings from a microphone sensor which measure nasal air pressure (NAP). NAP is measured by pressure transducer devices. NAF is measured by temperature sensing devices such as thermistors. NAP and/or NAF devices are used to measure OSA, mixed, central and hypopnea events because OSA and Hypopnea are defined as breath measured.

An example, a device has one microphone situated at the outlet of one nostril. An example, a device has two microphones, one situated at the outlet of each of the left and right nostrils, the data from the two microphones being combined into one real-time stream at identical sample rates and bits per sample.

An example, a device has one or more microphone situated on the nose, facing the surface of the skin, detecting NAP or NAF by measuring the audio signal through the skin. An example, a device has one or more microphone situated on the nose, facing away from the surface of the skin, detecting NAP or NAF by measuring ambient audio mixed with audio from the subject's breathing.

An example, a device has a combination of microphones facing toward the skin and away from the skin, combining their audio measurements to determine NAP and reduce the influence of ambient noise. An example, a device has one or more microphone situated near the mouth of the subject, measuring audio from the subject's oral breathing as well as a contribution of the subject's nearby nasal breathing, these measurements combined to determine NAP. An example, a device uses one or more model, through which the audio data values are passed, each model in series and/or in parallel selected to reduce the influence of background noise in the accuracy of actual NAP measurements. The model processes the data in real-time through one or both of the time domain and the frequency domain.

An example, a device uses feedback paths to affect the configuration or settings in the one or more sensors by rating the quality of the processed audio data after passing through one or more models. The feedback path allows the device to select the optimum sensor configuration and/or settings for each sensor, for each subject, for each use, thereby to calibrate the data processing path on the device and output on the final stage a stream in real-time of optimized audio values used to determine NAP or NAF and other sleep disturbance measurements.

An example, the feedback path or paths may pass back and forth across the communication channel between the Device 5110 and the Smart Device 5150, one or both of the subsystems participating in the processing of data and calibration of the sensors.

The data collected on the Device 5110 from the one or more sensors and processed through the one or more models and provided to the one or more feedback paths is used to determine breathing events. Each breath is recognized. Its magnitude, duration of inhale and exhale, delay from the preceding breath, distinguishing noise characteristics such as gasping or snoring are all determined in real time using the data from the one data stream. Using models, calibration, machine learning and feature recognition, all the necessary parameters of the subject's breathing are determined by the single, real-time data stream from the device.

An example, formants are recognized as features of each breath as a part of the data processing pipeline, these formants then being used to characterize each breath as open, obstructed, partially obstructed, recuperative, and similar aspects.

Sleep data is analyzed in real time and distinguishes obstructive apnea events from central and mixed apnea events. The system may then signal or through other mechanisms trigger the intervention of other equipment, such as CPAP, hypoglossal nerve stimulation, genioglossus muscle stimulation, or other pharyngeal or thoracic stimulation, only for obstructive or mixed apnea events and not for central apnea events. All types of apnea events are reported to the subject at the end of a sleep period.

Data on other biometrics is gathered and correlated to pre-existing conditions of the subject, thereby allowing the system to signal or announce particular apnea or hypopnea events that in real time may exacerbate one or more co-morbidities. Examples include blood pressure, heart rate, limb motion, preexisting medical conditions. Using real-time analysis, the system monitors the biometric data and issues warnings or alarms to the subject, to a sleep partner, or to an attending person such as a medical professional.

Form Factor

Apnea monitoring devices have limited success in part due to the level of discomfort and/or inconvenience in their use.

The Device 5110 form factor allows temporary attachment to the skin yet does not obstruct the nose or mouth and is not uncomfortable when worn through the course of a sleep period.

The Device 5110 shape is selected to minimize the bulk of the device on the skin while also placing all sensors in optimum locations. The shape also helps the subject to un-package, place, and remove the Device from the skin.

An example, a device shape includes one or more tabs, each tab helping the subject to place the device on the skin or to remove the device from the skin.

Measuring audio during sleep is a challenge which requires sensors to be placed close to the nose and/or mouth. The nose and mouth must remain unobstructed even when a measuring device is attached to the subject. These constraints create limitations and problems with such devices which attach by wire to the data recording subsystem, or which require large circuitry which does not fit into a device on the nose or adjacent to the mouth, or which is uncomfortable for the subject and does not allow for undisturbed sleep.

Data Collection

In some examples, analysis of measurements from one or both of the Smart Controller 120 and the Respiration Monitoring Device 120 may be performed by processing in a remote server, in the cloud, or on a computer separate from the Smart Controller but local to the User, such as a personal computer.

In some examples, the Obstructive Sleep Apnea Detection and Stimulation System 100 measures the User's sleep schedule over a period of days or weeks or longer, noting the clock time when the User begins the sleep period and the clock time when the User wakes during or at the end of the sleep period. The system analyzes this data and determines the most effective clock times to activate the Obstructive Sleep Apnea Detection and Stimulation System.

In some examples, Obstructive Sleep Apnea Detection and Stimulation System 100 collects time-based records of a User's sleep. These records are entered into a database of anonymized sleep period information from large populations of Obstructive Sleep Apnea Detection and Stimulation System Users, or with recordings of sleep periods from other detection systems.

In some examples, the OSA Detection System 100 uses AI techniques such as pattern recognition and correlation analysis to correlate real-time data recordings of the User with larger population databases to produce comparative or predictive analyses. In some examples, machine-learning algorithms are employed to build up the User's sleep history and provide specific predictors of sleep apnea severity and associated conditions.

In some examples, the time-based records of sleep periods are supplemented with data entered manually by one or more observer of the User's sleep.

The data recorded in the time-based database is sent to the cloud through a local network, such as a home mesh network, or directly over the Internet.

The apnea events counted in an AHI include obstructive, central and mixed apnea events. Similarly, hypopnea events of all types are counted into the AHI. This ratio of events per hour simplifies the assessment to a single number but takes no account of the differences in counts for apnea and hypopnea. A subject with a high hypopnea count and low apnea count will have an AHI matching that of a subject with a high apnea count and a low hypopnea count, provided that the total number of events per hour is the same. Since apnea and hypopnea have different effects on the subject, this single number over-simplifies the medical assessment.

The system and method calculate an AHI value and/or a SDI value continuously, such as hourly, during the sleep period and reports this metric and others across a timeline in real time in addition to an AHI metric and/or a SDI metric for the overall sleep period.

An example, when the data from the one or more sensors is determined to be of insufficient quality to be meaningfully processed for sleep disturbances, the system alerts the user by audible or visible processes. The user may choose to record again.

An example, the sensor data is scaled by mathematical processes such that the minimum and maximum post-scaling values are within a set minimum and maximum limit.

An example, the sensor data is filtered by mathematical processes such that the influence of background noise or signal noise is reduced, thereby improving the signal-to-noise ratio in the remaining sensor data.

An example, the background noise is separated from the sensor data values by filtering in one or more frequency bands. The background noise may be further characterized by matching to known background noise sources. Through the use of this matching process, optimized signal processing steps may be selected for a particular background noise source and those processing steps then applied continuously to the original sensor data to remove the effects of the background noise. Examples of such background noise are traffic noise, appliance noise near the user, electric fans, air conditioners, sixty cycle hum, fifty cycle hum, noise from movement of the bedding, noise from movement of the user or a sleeping partner.

An example, the optimized settings for filtering according to deduced background noise sources are stored in a database which is accessed in real time by the system.

An example, the sensor data characterization and qualification is performed continuously through the duration of the sleep session.

An example, the sensor data characterization and qualification is performed for a set period at the beginning of the recording, such as before the user settles into a sleeping position, or before the user falls asleep, or after a set number of minutes. By performing the qualification before sleep, the system alerts the user that the background noise is too much for proper analysis of the sleep data. By performing the qualification before the longer sleep period, the resources in the system needed for such qualification are used only during the qualification period, thereby extending the memory usage and/or the battery life of the system to last through the length of the sleep session.

Additional Sensors

The existence of “Apnea’ and ‘hypopnea’ center around the limitation in airflow during an attempted breath. An apnea event is marked during PSG as a reduction in airflow of at least 90% for a duration of 10 seconds or greater. A hypopnea event is marked as a reduction in airflow volume of at least 30% for a duration of 10 seconds or greater, that allows for a hypopnea event to be marked up to 89% reduction in airflow, thus allowing for misinterpretation of one or more event by the somnography technician.

In addition to tracking respiration using airflow to fulfill the criteria for ‘apnea’ and ‘hypopnea’ events, the system and method track multiple other biometrics in real time.

Examples of additional biometrics include one or more of the following: oxygen saturation primarily measured by pulse oximetry (SpO2), arterial oxygen saturation (SaO2), body temperature, heart rate, heart rate variation, electroencephalogram (EEG), electrocardiogram (ECG), electrooculography (EOG), or actigraphy.

An example, the system estimates one or both of SpO2 level and SaO2 level, and uses one or both in the data analysis pipeline.

An example, NAP and NAF exhaled breath is analyzed chemically with sensors to determine chemical compositions that will be indicative of disease states in the subject (Breathomics), for example VOC (volatile organic compounds) sensors coupled with the audio NAP/NAF sensors. VOCs are produced in various physiological and pathophysiological conditions thus making it a potential biomarker for several diseases. Coupling the VOC analysis with breath pattern analysis yields insights as to disease states of the subject.

An example, the NAP/NAF breath analysis is coupled with stimulation of the phrenic nerve to provide therapy in certain respiratory situations, since the phrenic nerve controls the movement of the diaphragm.

An example, NAP/NAF/SpO2 analysis adds more important information to the subject monitoring task using the four vital signs (body temperature, pulse rate, respiration rate, blood pressure) which are routinely monitored in the hospital and in critical care settings. Respiration rate alone does not tell very much about the condition of the respiratory system nor whether there are problems existent or incipient.

An example, real-time analysis of the respiratory system will be invaluable in the critical care environment when the subject is being ventilated. The combination of ventilation with breath analysis, with or without apnea, provides improved care.

The system and/or method uses one or more machine model to analyze the sleep data and other biometric data by machine learning. This learning relies on a database of large population sleep data. Each subject's system is adjusted through programmable processes to optimize the operation of the machine learning neural network using parametric settings determined through a priori analysis of earlier subject sleep sessions and similarities to a portion of the individuals in the population-based machine learning model. Thus, each subject's sleep session is monitored by the system using machine learning and calculation-based analysis, each tuned to parameter settings for that subject. By using machine learning as an expert system, the System is able to calculate results and deduce patterns in the data which a human observer is not able to recognize, the outputs of which affect the subject's sleep behavior. For example, a PSG human expert may recognize apnea events effectively with multiple data sources, such as the eight common signals from a sleep study, while the System here is able to recognize the same apnea events with only one data source, such as a microphone recording audio data.

An example, the system and/or method uses one or more optimization searches to find the parameter settings for each machine learning model, such as for example variance and normalization, to build a customized model for each user.

An example, the system includes sensors for other biometrics. Examples include SpO2 sensor, carbon monoxide and/or other chemical sensor, heart beat sensor, ECG sensor, body temperature sensor, accelerometers for body position sensing and/or limb movement, skin impedance sensor, environment temperature sensor, humidity sensor.

An example, the system uses an approximation of the subject's SpO2 level by analyzing the real-time breathing data based on NAP and/or NAF data using one or both of calculation-based processes and machine learning processes to deduce a blood oxygen level estimate. This estimate is adequate to mark periods when low blood oxygen levels affect adversely the health of the subject (hypoxia, hypercapnia), without the need for a specific, direct sensor-based measurement of blood oxygen level.

The combination of apnea plus SpO2 desaturation events can lead to the pathophysiology of apnea to IH (Intermittent Hypoxia); IPS (Intrathoracic Pressure Swings); and SF (Sleep Fragmentation due to arousals).

An example, the system and/or method uses one or more optimization searches to find the parameter settings for each machine learning model and for one or more of the additional signals brought in to the system, for example SpO2.

The system determines through a comparison and analysis of the subject's sleep data to the population database machine model whether the subject's sleep is normal or anomalous, and if non-normal, signals or announces to the subject, a sleep partner or an observer such as a medical professional that the sleep is unusual. This signaling and/or announcing may occur during the course of the sleep period while the system monitors and analyzes the sleep data in real time. For example, a message or alarm may be sent to a doctor or other medical service in real time that may trigger human activity if the monitored condition exceeds certain predetermined limits.

User Interface

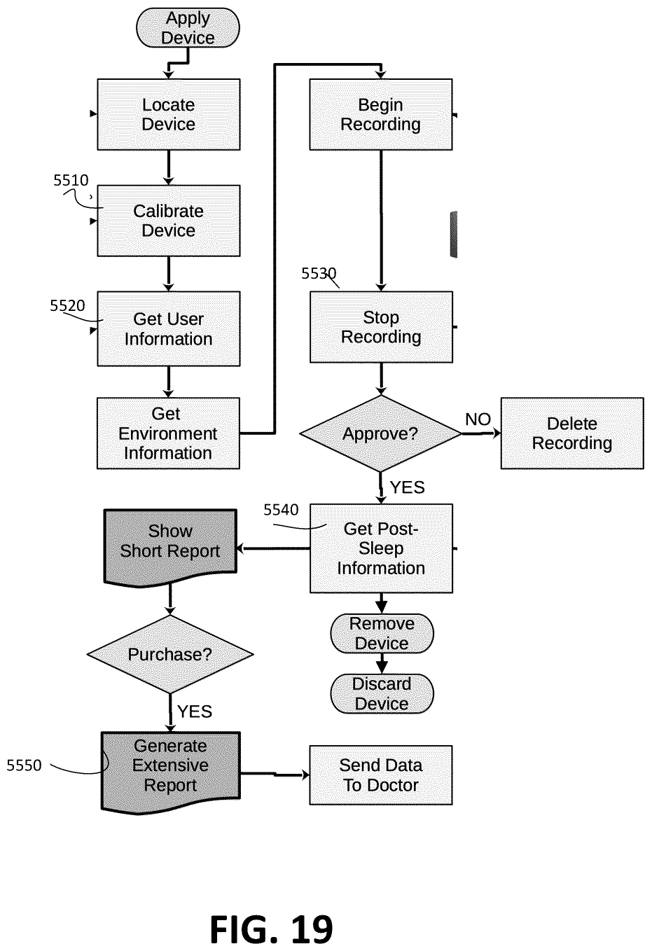

FIG. 19 shows an example flowchart for a user interface on the Smart Device. A device (e.g., patch 5110) is placed on the user and located by a smart device, for example by establishing a connection over wireless link such as BLE. The device is then calibrated 5510 under control of the smart device and/or a processor on the device. The smart device solicits information from the user and about the sleeping environment 5520. The user initiates a sleep recording using the user interface on the smart device, the recording proceeding until paused, resumed or stopped by the user 5530. The smart device asks the user to approve the recording, which may have been disturbed by extraneous noise such that the recording is not useful. The smart device asks the user for information after sleeping 5540, for example presence of a sleep partner. The smart device then directs the user to remove and discard the device 5540. After collecting the post-sleep information, sending the sleep recording for sleep disturbance analysis, and receiving statistics back from such analysis, the smart device displays or delivers to the user a short report 5550, summarizing the analysis. The user is given the opportunity to send an extensive report to their medical professional for later consultation.

An example, the recording is analyzed after the sleep period concludes and before being analyzed for sleep disturbances, this analysis serving to qualify the recording as to its suitability for subsequent analysis. Extraneous noise, interruptions to sleep, malfunctions may be disqualifying aspects of the recording. Recordings which do not measure up to the necessary quality are discarded.

An example, the post-sleep signal quality analysis is performed on one or more of the device, the smart device, a remote computer.

An example, sleep recordings with poor quality as assessed are broken up into shorter recordings, the acceptable quality portions collected into one group, and the unacceptable quality portions discarded. The acceptable time segments are annotated in parallel with the audio data, such that sleep disturbance analysis can compute statistics even with discontinuous recorded data.

An example, the user pays a fee to deliver the extensive report 5550 to their medical professional.

An example, the fee is paid by the user each time a sleep report is to be delivered to the medical professional.

An example, the fee is part of a subscription paid by the user to cover one or more sleep period recordings.

An example, the user receives the extensive report.

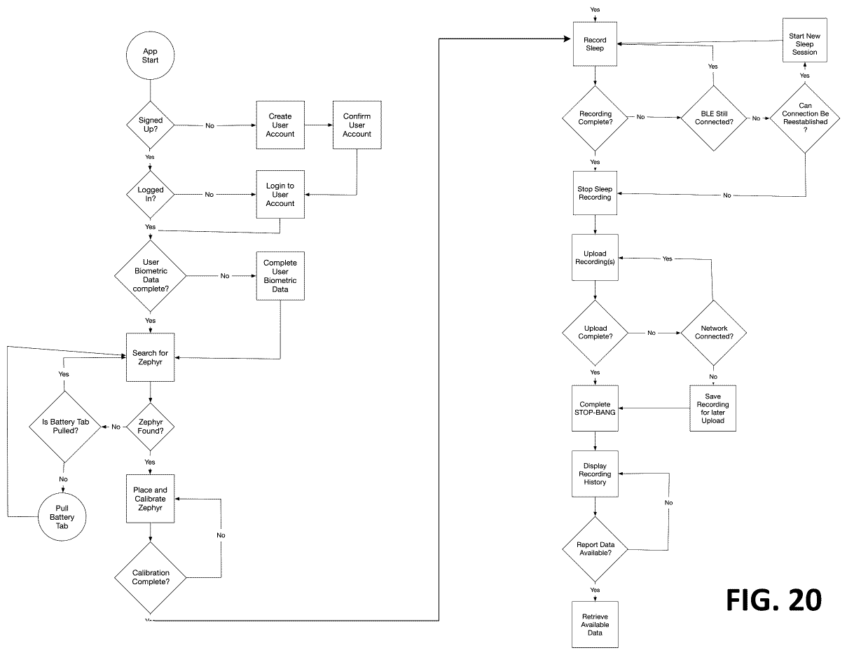

FIG. 20 shows an example flowchart for a user interface on the Smart Device including user log-in. A smart device requests from a user a log-in name and password that are used to protect the user's information and recorded sleep data.

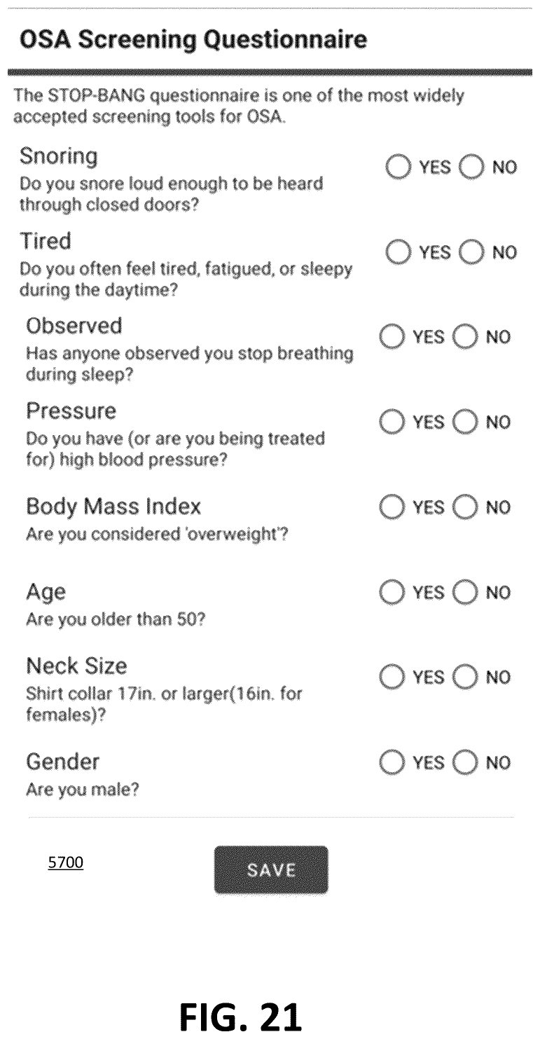

FIG. 21 shows an example Subject Information User Interface 5700.

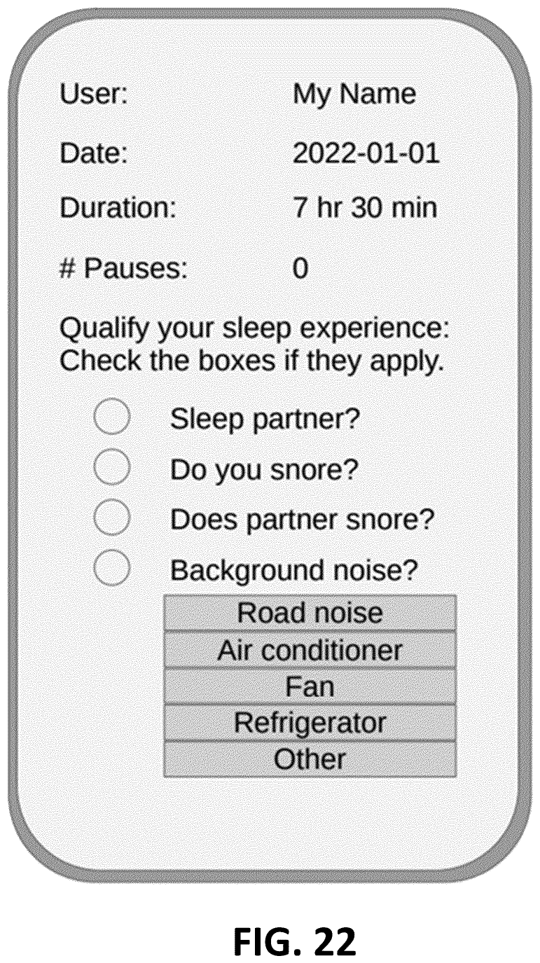

FIG. 22 shows an example Sleep Situation Information User Interface 5800.

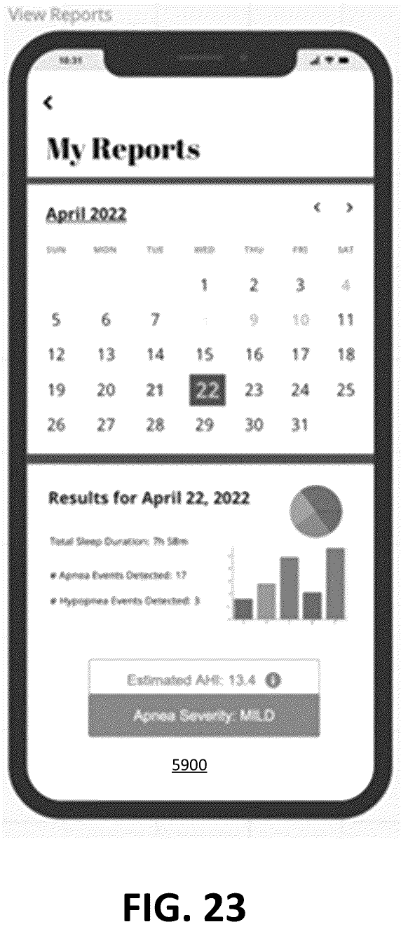

FIG. 23 shows an example After Sleep Short Report 5900.

The UI 5700 is presented to the subject. Through this interface, the subject enters information, including personal information such as weight, BMI, and situational information related to the sleep period such as the presence of background noises, the presence of a sleep partner, the starting sleep position, and results from the sleep period analysis are presented to the subject.

An example, a system uses its one or more sensors and a processor and/or a smart device to calibrate the collection of data. For example, a short recording of ambient audio is collected, then processed, then the settings and adjustments on the one or more microphones are changed to optimize the quality of the input data. Additionally, the parameters of data processing are adjusted according to the sampled calibration data, such as noise filters, frequency domain settings, scaling values, machine learning parameter settings.

An example, a UI provides the subject with the ability to start, pause, end, discard and save a sleep data recording.

An example, a UI provides the subject information on the quality of the data collected.

An example, the data is collected for a period before the subject applies the device to the skin. The collected data is analyzed on the device and/or on a smart device, and a determination made as to whether the environment intended for the sleep recording is appropriate for a meaningful analysis of the later sleep data. For example, there may be too much background noise, whether continuous or occasional, to allow for quality recording from the sensor or sensors on the device. For example, there may be too much noise from a sleep partner to allow the device and/or the smart device to distinguish the sleep data of the subject from that of the sleep partner. In each case, the UI provides this assessment to the subject and the subject is provided the means on the user interface to begin or not begin a recording.

An example, the data is collected for a period after the subject applies the device to the skin but before beginning to sleep. As in the aforementioned case, various circumstances may constrain a device from collecting meaningful data. As before, the subject is presented with the means to begin or not begin a recording.

An example, the data is collected in one or more segments through the course of one sleep period, the subject having affixed a device to the skin and initiated a recording using the means provided on a UI. During or after all or part of the sleep session, the Device and/or a smart device assesses the quality of the collected or calculated data and informs the subject what it determines to be the quality of the recording. The subject may choose to continue recording or to discard some or all of the data from the sleep session, using means provided on the UI.

An example, a smart device provides the means for the subject to selectively delete portions of the sleep session recording or recordings, choosing those portions in which noise or system malfunction created unusable data. The indication of such unusable data is presented on the UI by the smart device. For example, the subject may be aware of specific times when environmental noise intrudes or may intrude on the recording, such as chiming of a clock at the hour, the passing of a train, the ringing of a doorbell.

An example, a smart device provides the means for the subject to pause and resume the ongoing recording during a sleep session, such as when the subject wakes to leave the bed and use the bathroom.

An example, the pause and resume actions do not stop and restart the actual sensor data recording, but rather create annotations in one or more files on a smart device, or on an auxiliary, metadata stream from the device to the smart device, the annotations indicating stop and resume clock times or times along the measured timeline of the recording.

An example, the pause and resume action stop the recording of sensor data and then resume the recording of sensor data, the timeline for the recorded data indicating breaks in the continuity of the timeline.

The UI 5900 provides the subject information on the calculations performed on the collected data, the information being displayed on a Smart Device, and/or able to be delivered to another device with more display area, and/or able to deliver to a printing device to make a physical record of the calculations.

An example, the calculations include measures of the quality of sleep of the subject during the recorded sleep session. Measurements may include AHI, SDSI, running AHI (calculated on a running basis through the course of the sleep session), sleep stage, breath cadence, snoring, sleep movement, departures from sleep such as using the bathroom, interruptions to sleep such as coughing, and similar metrics. Any of these measurements may be presented in a scale which indicates the severity of the measurement, from ‘normal’ to ‘severe’, such that the subject may change their sleep behavior or may pursue additional medical advice. For example, if AHI is shown to be moderately severe but only during portions of the sleep session when the subject slept on their back, then the subject may choose different bedding options to keep from sleeping on their back. For example, based on data input by the subject each night before sleeping, the subject may choose after viewing the UI report that nights when they had had a large meal, or eaten later in the evening than usual, lead to worse AHI that they should avoid such behavior.

An example, a UI may present the subject with suggestions to seek additional medical advice based on the severity calculated for one or more sleep indicators.

An example, when suggesting additional medical advice, a UI may provide the means for the subject to transmit their sleep data and/or the calculations and assessments to a medical professional of their choosing. When making such a transfer of information, the system abides by all necessary requirements for data security and privacy.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of detecting and treating an occurrence of an apnea event during a sleep period of a user, the method comprising:
receiving data corresponding to nasal air pressure of the user and/or audio of the user during the sleep period;
determining patterns in the data;
based on the patterns, detecting the apnea event;
in response to the detecting, treating the apnea event.

2. The method of claim 1, the treating comprising using an external hypoglossal nerve stimulator system.

3. The method of claim 1, the treating comprising using an internal hypoglossal nerve stimulator system.

4. The method of claim 1, the treating comprising using a positive airway pressure device.

5. The method of claim 1, the determining patterns in the data comprising training a machine learning model using input polysomnography (PSG) recordings.

6. The method of claim 5, further comprising:
based on the patterns, determining an SpO2 level over the sleep period in using the trained machine learning model.

7. The method of claim 5, further comprising:
using the trained machine learning model to predict one or more comorbidity ailments of the user in response to detecting the apnea event and biometric data corresponding to the user.

8. The method of claim 1, the receiving data comprises affixing a patch externally on a dermis of the user, the patch comprising a flexible substrate, an adhesive on a first side adapted to adhere to the dermis of the user, a processor directly coupled to the substrate, and one or more sensors directly coupled to the substrate.

9. The method of claim 8, the patch in wireless communication with a smart device.

10. A sleep system for detecting and treating an occurrence of an apnea event during a sleep period of a user, the system comprising:
one or more sensors for receiving data corresponding to nasal air pressure of the user and/or audio of the user during the sleep period; and
one or more processors that determine patterns in the data and based on the patterns, detecting the apnea event and, in response to the detecting, treating the apnea event.

11. The sleep system of claim 10, the treating comprising initiating an external hypoglossal nerve stimulator system.

12. The sleep system of claim 10, the treating comprising initiating an internal hypoglossal nerve stimulator system.

13. The sleep system of claim 10, the treating comprising initiating a positive airway pressure device.

14. The sleep system of claim 10, the determine patterns in the data comprising training a machine learning model using input polysomnography (PSG) recordings.

15. The sleep system of claim 14, the one or more processors further comprising:
based on the patterns, determining an SpO2 level over the sleep period in using the trained machine learning model.

16. The sleep system of claim 14, the one or more processors further comprising:
using the trained machine learning model to predict one or more comorbidity ailments of the user in response to detecting the apnea event and biometric data corresponding to the user.

17. The sleep system of claim 14, further comprising:
a patch adapted to be affixed externally on a dermis of the user, the patch comprising a flexible substrate, an adhesive on a first side adapted to adhere to the dermis of the user, a processor directly coupled to the substrate, and one or more sensors directly coupled to the substrate.

18. The sleep system of claim 17, the patch in wireless communication with a smart device.

19. A non-transitory computer readable medium having instructions stored thereon that, when executed by one or more processors, cause the processors to detect and treat an occurrence of an apnea event during a sleep period of a user, the detecting and treating comprising:
receiving data corresponding to nasal air pressure of the user and/or audio of the user during the sleep period;
determining patterns in the data;
based on the patterns, detecting the apnea event;
in response to the detecting, treating the apnea event.

20. The non-transitory computer readable of claim 19, the determining patterns in the data comprising training a machine learning model using input polysomnography (PSG) recordings.
